(19) United States
(12) Patent Application Publication
(10) Pub. No.: US 2009/0239875 A1
(43) Pub. Date: Sep. 24, 2009

(54) Protection of ferulic acid and/or tetramethylpyrazine against retinal ischaemia, glaucoma and siderosis oculi

(75) Inventor: Hsiao-Ming Chao, Taipei (TW)

Correspondence Address: WPAT, PC;INTELLECTUAL PROPERTY ATTORNEYS, 2030 MAIN STREET, SUITE 1300, IRVINE, CA, 92614, US

(73) Assignee: Committee on Chinese Medicine and Pharmacy, Department of Health, Executive Yuan, Taipei City (TW)

(21) Appl. No.: 12/053,425

(22) Filed: Mar. 21, 2008

(51) Int. Cl. A61K 31/4965; A61K 31/192; A61P 25/28

(52) U.S. Cl. 514/252.1; 514/570

(57) ABSTRACT

A method for preventing and/or treating ischaemic and/or iron-related retinal or brain disorders comprising administering an effective amount of ferulic acid (FA), tetramethylpyrazine (TMP) or their pharmaceutically acceptable salt, ester, solvate, hydrate, analogs, metabolite, enantiomer, isomer, tautomer, amide, derivative or prodrug to a subject. The former diseases comprise retinal ischemia, glaucoma as well as brain ischaemia (i.e. stroke, infarction typed). The latter ones comprise age-related macular degeneration, intraocular hemorrhage, siderosis oculi (due to retained intraocular iron), oxidative stress of the retina as well as brain hemorrhage (stroke, hemorrhagic type) or Alzheimer disease. Clinically, FA alone or in combination of TMP can be administered systemically, orally, intravitreously, topically (in form of eyedrops), as well as other routes such as periocular, subconjunctival, and intracamera.

### FIELD OF THE INVENTION

The present invention is related to a pharmaceutical therapy for preventing or treating retinal ischaemia, glaucoma, ocular hemorrhage and AMD.

### BACKGROUND OF THE INVENTION

Central retinal artery and vein occlusion, diabetes, and glaucoma are conditions that can be associated with ischemic changes of the retina. Clinically, retinal ischemia is recognized by an alteration in the b-wave of electroretinogram (ERG), cotton wool spots, hemorrhage, and sometimes pathologic disc “cupping”. Worldwide, there are 65 million people with glaucoma of whom around 7.5 million have become blind (Quigley, H. A. Br. J. Opthalmol. 80:389-93, 1996). Furthermore, it is the second most common cause of blindness in the world. Even in the developed world, where attempts at early diagnosis are established, there is little sign that the problem is diminishing. This indicates that glaucoma is an intractable problem that needs to be urgently managed with. Glaucoma and retinal ischaemia, such as central retinal artery occlusion (CRAO), share a number of pathological similarities (Quigley, H. A. Prog. Retin. Eye Res. 18(1):39-57, 1999). CRAO, for example, is an ocular emergency that not uncommonly ends with a poor visual outcome if treatment is delayed longer than 97 minutes (Hayreh, S. S., et. al. Exp. Eye Res. 78:723-36, 2004). Ischaemic insult is also known to play an important role in diabetic retinopathy. In addition, oxidative stress, a prominent event in the ischaemic cascade, is strongly associated with age-related macular degeneration (A M D; Zarbin, M. A. Arch. Opthalmol. 122:598-614, 2004; Dunaief, J. L. Invest Opthalmol Vis Sci. 47(11):4660-4, 2006 November). Therefore, like glaucoma, the management of retinal ischaemia and AMD appears to be a critical issue as well.

It is believed that neurones expressing glutamatergic receptors are vulnerable to ischaemia/reperfusion (Osborne, N. N., et. al. Prog. Retin. Eye Res. 23:91-147, 2004). Furthermore, these neurones such as retinal ganglion cells (RGCs) and amacrine cells as well as their neuronal processes are known to be located in the inner retina. Moreover, choline acetyltransferase (ChAT) immunopositive cholinergic amacrine neurones are located in the inner nuclear layer (INL) and the ganglion cell layer (GCL) and their neuronal processes give a characteristic 2-strata pattern in the inner plexiform layer (IPL; Chao, H. M., et. al. Brain Res. 904:126-136, 2001). What is more, it has been revealed that the drastic reduction in the levels of Thy-I mRNA and protein caused by pressure-induced retinal ischaemia are associated with RGC damage (Ahmed, E, et al. Invest. Opthalmol. Vis. Sci. 45(4):1247-58, 2004). Excess production of reactive oxygen species (ROS) has been reported after ischaemia/reperfusion6 and, as a consequence, any drug that has the capacity to inhibit formation of ROS such as hydroxyl radicals in stressed cells may be a potential neuroprotectant.

It has been previously shown that Ligusticum walliichi (Chuan Xiong) has long been used by practitioners of traditional Chinese medicine in treating blood stasis and consequent energy insufficiency/depletion (Ho, W. K., et al. Stroke 20:96-99, 1989). In recent years, two active ingredients of this herb have been characterized and its chemical structure has been worked out, namely ferulic acid (FA) and tetramethylpyrazine (TMP). TMP, an alkaloid, has been reported to be effective for the treatment of brain/spinal cord ischaemia (Kao, T. K., et al. Neurochem. Int. 48:166-76, 2006; Fan, L. H., et al. BMC Neurosci. 14:48, 2006) and H₂O2-induced apoptosis (Cheng, X. R., et al Cell Biol. Int. 31:438-43, 2007). Polyphenols, such as FA, occur ubiquitously in plant kingdom. They are important for normal growth development and defense against chronic neurodegeneration such as Alzheimer's disease (Mohmmad Abdul, et al Biochim. Biophys. Acta. 1741(1-2):140-8, 2005). In other previous reports using in vitro culture systems, FA has been shown to act as an antioxidant by forming a resonance stabilized phenoxy radical and scavenged free radicals such as hydroxyl radicals (Kanski, J., et al. J. Nutr. Biochem. 13:273-281, 2002).

Siderosis bulbi is vision-threatening. An investigation into its mechanisms and management is crucial. Experimental siderosis was established by intravitreous administration of an iron particle (chronic), or FeSO4 (acute). After siderosis, there was a significant and/or dose-responsive reduction in eletroretinogram (ERG) b and/or a-wave amplitude, and increase in .OH level, which was greater caused by 24 mM FeSO4 than that by 8 mM. Furthermore, the oxidative stress induced by 8 mM FeSO4 was significantly blunted by 100 mM FA. The siderosis also resulted in an excessive glutamate release, an increased [Ca⁺⁺]i, together with enhanced superoxide dismutase immunoreactivity. The latter finding was consistent with the Western blot result. Obvious disorganization including loss of photoreceptor outer segments and cholinergic amacrines, together with a wide-spreading ferric distribution across the retina was present. The eletro-retinographic and pathologic dysfunctions were respectively significantly/dose-dependently, and clearly ameliorated by FA. Conclusively, the siderosis stimulates oxidative stress, and, possibly, subsequent excitotoxicity, and calcium influx. This explains why the retina is impaired electro-physiologically and pathologically. Importantly, the iron toxicity is protected by FA via partly acting as free radical scavengers. This invention provides an approach in treating the iron-related disorders such as intraocular hemorrhage, AMD and Alzheimer disease.

Iron deposition, oxidative stress, or mitochondrial insufficiency may play a role in the ageing and degeneration of the central nervous system (Schipper, H. M. Brain iron deposition and the free radical-mitochondrial theory of ageing. Ageing Res. Rev. 3(3):265-301, 2004). In the brain, iron may also contribute to the pathogenesis of neuro-degeneration by promoting oxidative damage (e.g. Alzheimer's disease, Parkinson's disease, etc; Levine, S. M. Iron deposits in multiple sclerosis and Alzheimer's disease brains. Brain Res. 760(1-2):298-303, 1997 June; Kaur D., Anderson J. Does cellular iron dysregulation play a causative role in Parkinson's disease? Ageing Res. Rev. 3(3):327-343, 2004 July). In advanced ocular siderosis, retinal circulatory insufficiency, or oxidative stress presumably contributes to the narrowed visual fields together with reduced electroretinographic (ERG) and electroculographic (EOG) functions (Chao, H. M. et al. Siderosis Oculi: visual dysfunctions even after iron removal; a role of OCT. Cutan. Ocul. Toxicol. 25(2):131-140, 2006) Clinically, retained intraocular iron is a vision-threatening event and these events include iron intraocular foreign body (IOFB) (Chao H M et al. Siderosis Oculi: visual dysfunctions even after iron removal; a role of OCT. Cutan. Ocul. Toxicol. 25(2):131-140, 2006) and submacular hemorrhage in myopes without choroidal neovascularization (manuscript in preparation). This might also be the case in the proliferative diabetic retinopathy associated with vitreous hemorrhage (iron in hemoglobin), which usually take months to clear. Moreover, iron induced oxidative damage appears to be a potential factor in age-related macular degeneration (Dunaief, J. L. Invest Opthalmol V is Sci. 47(11):4660-4, 2006 November). Above all, the mentioned victims are generally distributed among productive ages, and not in a small number in Asia or other developing countries. Their misfortunes are tragedies to their family and traumatic to the whole society. In the present invention, it is therefore a vital step in the investigation of the underlying mechanisms of iron-induced retinal toxicity and eventually may result in more effective management of the toxicity.

Iron powder (Fe0) & ferrous ion (Fe2+) resulted in retinal damages. Furthermore, these injuries are much more severe than those occurring after administration of ferric ion. This provides evidence to support the toxic effect of the iron oxidation on the retina.

It has been widely acknowledged that O2.- is produced during normal aerobic metabolism through several pathways. Superoxide dismutase (SOD) converts O2 into H2O2. The latter two components are known to be the essential substances for the Fenton reaction. The Fenton reaction, in a living tissue such as the retina, is able to cause iron oxidation, which stimulates the formation of highly reactive oxygen species (ROS), e.g. .OH (Chakraborti, T. et al. Oxidant, mitochondria and calcium: an overview. Cell Signal 11: 77-85, 1999). In the present invention, a comparison between the injuries caused by iron particle (chronic) and FeSO4 (acute) was first made. It is known that free radicals can enhance neuronal glutamate release (Pellegrini Giampietro, D. E. et al. Excitatory amino acid release and free radical formation may cooperate in the genesis of ischemia-induce neuronal damage. J Neurosci 10:1035-1041, 1990) and inhibit glutamate uptake by glial cells (Volterra A et al. Glutamate uptake inhibition by oxygen free radicals in rat cortical astrocytes. J Neurosci 14:2924-2932, 1994). It has also been demonstrated that the levels of glutamate are elevated in iron-induced cerebral (Engstrom, E. R. et al. Extracellular amino acid levels measured with intracerebral microdialysis in the model of posttraumatic epilepsy induced by intracortical iron injection. Epilepsy Res 43: 135-144, 2001) or retinal degeneration. Calcium influx might be subsequently stimulated through, at least in part, ligand-gated calcium channels. It is logical that oxidative stress, excitoxicity, and calcium influx may explain iron-induced retinal toxicity. This invention aims at providing direct evidences linking defined biochemical markers with iron toxicity in the retina.

It has been reported that a phenolic compound FA possessed anti-oxidative effect and might thus be capable of scavenging O2 and .OH. (Srinivasan M et al. Influence of ferulic acid on gamma-radiation induced DNA damage, lipid peroxidation and antioxidant status in primary culture of isolated rat hepatocytes. Toxicology 7228(2-3):249-258, 2006 December; Zhang Z et al. Iron-induced oxidative damage and apoptosis in cerebellar granule cells: attenuation by tetramethylpyrazine and ferulic acid. Eur J Pharmacol 467(1-3): 41-47, 2003 Apr. 25). So did TMP have free radical scavenging effect (Zhang Z et al. Iron-induced oxidative damage and apoptosis in cerebellar granule cells: attenuation by tetramethylpyrazine and ferulic acid. Eur J Pharmacol 467(1-3): 41-47, 2003 Apr. 25). Presently, it will also be evaluated whether and, furthermore, how these “putative” neuroprotectants are able to protect the retina against the iron toxicity caused oxidative stress. Therefore, they possibly provide an Ann. Clin. Psychiatr additional non-surgical approach to curing or ameliorating iron toxicity in a number of brain and ocular disorders (Schipper H M. Brain iron deposition and the free radical-mitochondrial theory of ageing. Ageing Res Rev 3(3): 265-301, 2004; Levine S M. Iron deposits in multiple sclerosis and Alzheimer's disease brains. Brain Res 760(1-2): 298-303, 1997 June; Kaur D, Anderson J. Does cellular iron dysregulation play a causative role in Parkinson's disease? Ageing Res Rev 3(3): 327-343, 2004 July). There are, for example, chronic Alzheimer's disease, acute hemorrhagic stroke, cataract, glaucoma, ocular hemorrhage, and AMD (Dunaief, J. L. Invest Opthalmol V is Sci. 47(11):4660-4, 2006 November).

### SUMMARY OF THE INVENTION

The present invention provides a method for preventing or treating retinal or brain disease comprising administering an effective amount of ferulic acid, tetramethylpyrazine or their pharmaceutically acceptable salt, ester, solvate, hydrate, analogs, metabolite, enantiomer, isomer, tautomer, amide, derivative or prodrug to a subject, wherein the retinal disease comprising retinal ischemia, oxidative stress of the retina, glaucoma, age-related macular degeneration, or ocular hemorrhage, as well as brain ischaemia (i.e. stroke, infarction typed).

The present invention also provides a method for preventing or treating iron-related disorder comprising administering an effective amount of ferulic acid, tetramethylpyrazine or its pharmaceutically acceptable salt, ester, solvate, hydrate, analogs, metabolite, enantiomer, isomer, tautomer, amide, derivative or prodrug to a subject, wherein the iron-related disorder is retained intraocular iron, brain hemorrhage (stroke, hemorrhagic type) or Alzheimer disease.

### BRIEF DESCRIPTION OF THE DRAWINGS

FIG. 1. HPLC chromatogram (A, and B). The formation of hydroxyl radicals (.OH) was quantified by constructing a standard curve using various amounts of pure 2,3-DHBA (12-60 pmoles). Twelve pmoles (A; 15 μl of 0.8 μM 2,3-DHBA) is an example of the standard curve of 2,3-DHBA; Thirteen point one pmoles of 2,3-DHBA (B) was formed from 15 μl of a vitreous dialysate sample collected at 75 minutes of HIOP-induced retinal ischaemia. The peak as indicated at 18˜21 minutes (A and B) represents the compound, 2,3-DHBA, which is produced by the action of .OH on salicylic acid. HIOP, high intraocular pressure; HPLC, high performance liquid chromatography.

FIG. 2. Effects of HIOP-induced retinal ischaemia on the toxic hydroxyl radicals (.OH) formation in the rat retina. Production of .OH was quantified as the amount of 2,3-dihydroxybenzoic acid (2,3-DHBA) using HPLC-EC detection. Results are mean ±S.E.M of the number of the animals in the parenthesis. The formation of .OH was quantified by constructing a standard curve using various amounts of pure 2,3-DHBA (12-60 pmoles). The .OH formed was measured every 15 minutes (45 minutes before ischaemia, and during the 2-hour ischaemia). As compared with the sham-operation rats (control), the level of 2,3-DHBA in the ischemic rats gradually and significantly increased at 45, 60, 75 and 105 minutes of ischaemia, with a peak at 75 minutes. Significantly different (*p<0.05) from the sham-operation group by unpaired Student's t-test.

FIG. 3. Summary of the electroretinogram b-wave ratio results showing the effect of intravitreously applied vehicle (saline), 0.5/0.1 nmole ferulic acid (FA), or 0.5 nmole tetramethylpyrazine (TMP) on rats subjected to 60 min retinal ischaemia plus reperfusion (I/R) for 7 days. In comparison with the vehicle-treated group (control), FA significantly and dose-dependently attenuated the I/R-induced reduction in the b-wave amplitude 3, 5, or 7 days after I/R. In contrast, TMP had a slight but statistically insignificant attenuating effect. Results are mean ±S.E.M. for the number of animals shown in the parenthesis. Significantly different (**p<0.01; *p<0.05) from the vehicle-treated ischaemic group by one-way ANOVA followed by Tukey multiple-comparison test.

FIG. 4. Effect of pre-administration [60 minutes before ischaemia plus reperfusion (I/R) for 7 days] of different substances on the changes in ChAT immunoreactivity (red colour) induced by I/R. In the sham-operation (normal) retina, ChAT is associated with amacrine perikarya (short arrows; A) in the INL and GCL and their processes in the IPL, which are seen as two clearly defined strata of immunoreactivity (long arrows; A). I/R caused an almost complete obliteration of the ChAT immunoreactivity in the IPL and the number of ChAT positive amacrine cell perikarya was significantly reduced; the ischaemia-induced alterations were not affected by treatment with the vehicle (C). The effect of ischaemia/reperfusion was obviously nullified by intravitreous application of 0.5 nmole ferulic acid (FA; E; long arrows, two strata in the IPL; short arrows, cell bodies in the INL and RGC layer); while only a partial counteracting effect was created by 0.1 nmole FA (G; long arrow, a strata in the IPL; short arrow, a single cell body in the RGC layer). In contrast, the ischaemic insult was not affected by 0.5 nmole tetramethypyrazine (TMP; I). The retinal cellular nuclei in different groups (A, C, E, G, or I) were respectively counterstained with DAPI (blue colour). The respective merge images of ChAT and DAPI labeling was shown in picture B, D, F, H, or J. The abbreviations are: ONL, outer nuclear layer; OPL, outer plexiform layer; INL, inner nuclear layer; IPL, inner plexiform layer; GCL, ganglion cell layer; ChAT, choline acetyltransferase; DAPI, 4,6-diamidine-2-phenylindole dihydrochloride. Scale bar=50 μm.

FIG. 5. This figure shows a RT-PCR minigel analysis of RNA (4 μg) from rat retinal cells for the expression of Thy-1 and β-actin mRNAs (upper picture). Seven days after ischaemia/reperfusion (I/R), whole retinal extracts were isolated from the normal eyes (sham-operation; lane 1), or the ischaemic eyes (subjected to 60-minute HIOP) pre-administered (1 hour before ischaemia) with intravitreous vehicle (lane 2), 0.5 (lane 3)/0.1 nmole FA (lane 4), or 0.5 nmole TMP (lane 5). The results are representative of four animals from each group.

The effect of defined intravitreous compounds on the levels of Thy-1 and β-actin mRNAs was analyzed (lower picture). The data represent the ratio of Thy-1 to β-actin mRNA. β-actin is a house-keeping gene. The symbol of * or †, respectively, represents a significant difference (p<0.05) between the vehicle-pre-treated ischaemic group and the sham operation group, or between the vehicle-pre-treated ischaemic group and the 0.5 nmole FA-pre-treated ischaemic group by one-way ANOVA followed by a Tukey multiple-comparison test. Results are the means ±S.E.M. of four animals from each group. The abbreviations are: HIOP: high intraocular pressure; FA, ferulic acid; TMP, tetramethylpyrazine.

FIG. 6. The effect of pre-administration [60 minutes before ischaemia plus reperfusion (I/R) for 7 days] of vehicle or FA on the changes in Thy-1 immunoreactivity induced by I/R. In the sham operation (normal) retina, Thy-1 is associated with a broad band in the GCL and IPL (A). In contrast, seven days after 60 min of ischemia, there was an obvious reduction in the thickness of Thy-1 immunoreactivity in the ischaemic retinas pre-treated with vehicle (B). Moreover, although thinning of the inner retina occurred, the outer retina appeared to be relatively unchanged. The effect of ischaemia/reperfusion was obviously counteracted by intravitreous application of 0.5 nmole FA (C). The weak fluorescent staining associated with the INL, OPL and photoreceptor outer segments in the retinal sections is nonspecific binding (A, B, C). As shown in the bar chart (D), in comparison with the sham operation retinas, a significant reduction in the thickness of the Thy-1 immunolabeling associated with the GCL and the IPL is evident in the ischaemic retinas pre-treated with vehicle. Moreover, as compared with the ischaemic retinas pre-treated with vehicle, the pre-treatment of 0.5 nmole FA significantly attenuated the I/R-induced thinning of the Thy-1 immunoreactivity. The abbreviations are: ONL, outer nuclear layer; OPL, outer plexiform layer; INL, inner nuclear layer; IPL, inner plexiform layer; GCL, ganglion cell layer. Scale bar=50 μm. The symbol of * or †, respectively, represents a significant difference (p<0.05) between the vehicle-pre-treated ischaemic group and the sham operation group or between the vehicle-pre-treated ischaemic group and the 0.5 nmole FA-pre-treated ischaemic group by one-way ANOVA followed by a Tukey multiple-comparison test. Results are the means ±S.E.M. of the number of animals shown in parenthesis.

FIG. 7. Effects of intravitreous injection (i.v.i.) various compounds on hydroxyl radicals (.OH) formation stimulated by 60-minute HIOP-induced retinal ischemia. Levels of .OH was measured as those of 2,3-dihydroxybenzoic acid (2,3-DHBA) using HPLC-EC detection. In comparison with the sham-operation (control; n=4) rats, 60-minute ischemia induced a significant increase in the levels of .OH in the vehicle-treated ischaemic rats (n=5). As compared with the vehicle-treated ischaemic rats, 0.5 nmole FA (n=4) significantly attenuated the increased levels of .OH. In contrast, 0.1 nmole FA and 0.5 TMP exerted respectively a lower and a small attenuating effect. Values are given as mean ±S.E.M. The symbol of * or †, respectively, represents a significant difference (p<0.05) between the vehicle-pre-treated ischaemic group and the sham-operation group, or between the vehicle-pre-treated ischaemic group and the 0.5 nmole FA-pre-treated ischaemic group by one-way ANOVA followed by a Tukey multiple-comparison test. HPLC, high performance liquid chromatography.

FIG. 8. A. Oxidized iron removed from the vitreous humor of a rat subjected to experimental siderosis (28 days). B. The electroretinogram (ERG) of a normal eye (untreated) illustrating the amplitudes of the a- and the b-waves. When an iron particle was intravitreously implanted for 24 hours, both the a- and the b-wave amplitudes became almost flat. C. An acrylate chip did not affect the ERG b-wave ratio. In comparison with the acrylate chip group, there was a significant decrease in the b-wave amplitude ratio after iron particle implantation from 4 hours to 24 hours measured every 2 hours (*p<0.05; unpaired Student's t-test). D. The FeSO₄-induced reduction in the b-wave amplitude ratio occurred in a dose-responsive manner compared with the physiological saline group. The ratios were measured before treatment (0 hour) and over the first 12 hours at 2 hours intervals. The results are the mean ±S.E.M. for the number of animals shown in the parenthesis. The significantly different results (*p<0.05; *p<0.01) compared to the saline control were analyzed by one-way ANOVA followed by a Tukey multiple-comparison test.

FIG. 9. HPLC chromatogram (A, B, D and E) and the time-dependent levels of 2,3-dihydroxybenzoic acid (2,3-DHBA; C; F) after intraocular administration of iron and other materials. 16 (A) or 40 pmoles (D) are examples of the standard curve of 2,3-DHBA; 36 (B) or 91.23 pmoles of 2,3-DHBA (E) were formed 135 minutes or 20 minutes after iron implant or ferrous sulfate injection (8 mM FeSO₄), respectively. The peak as indicated at 18˜21 minutes (A; B; D; E) represents the compound, 2,3-DHBA, which is produced by the action of hydroxyl radicals (.OH) on salicylic acid. The iron particle-stimulated increase in 2,3-DHBA production was significant compared with the acrylate chip group (C; *p<0.05; unpaired Student's t-test). In the left upper corner of the picture C, the minimized plot was modified by deletion of one measure with a greater variability out of the original four measures. Moreover, the values of .OH formed were expressed as a percentage with the initial baseline data as 100%.

Likewise, the 24 or 8 mM FeSO₄-stimulated increase in 2,3-DHBA formation was significant and dose-dependently compared with the physiological saline group (F). Furthermore, in comparison with the siderotic rats (insulted by 8 mM FeSO₄) treated with saline, 100 μM FA significantly decreased the production of OH. The symbol * or †, respectively, represents a significant difference (p<0.05) between the 24 or 8 mM FeSO₄ treatment group and the saline treatment group or between the 8 mM FeSO₄ treatment group and the FeSO₄ plus FA treatment group by one-way ANOVA followed by a Tukey multiple-comparison test. In the right middle part of the picture F, the minimized plot was modified by deletion of two groups (24 and 0.8 mM FeSO₄) out of the original five groups. Results are the means ±S.E.M. of the number of animals shown in parenthesis. HPLC, high performance liquid chromatography.

FIG. 10. HPLC chromatogram (A; B) and the time-related glutamate levels (C). A. One concentration (5×10⁻⁸ M) was used to create the standard curve for glutamate; B. The glutamate concentration (7.22×10⁻⁷ M) was measured 20 minutes after ferrous sulfate treatment (8 mM). The glutamate peak occurred at 2˜3 minutes (A; B). C. The time course for glutamate after intraocular injection of different dosages of ferrous sulfate. Glutamate levels were measured by HPLC using a fluorescence detector before and after ferrous sulfate treatment. The increase in production of glutamate was significant compared to the physiological saline group. Results are the means ±S.E.M. of the number of the animals in parentheses. The symbol * represents a significant difference (p<0.05) between the 24 or 8 mM FeSO₄ treatment group and the saline treatment group by one-way ANOVA followed by a Tukey multiple-comparison test. HPLC, high performance liquid chromatography.

FIG. 11. Increased intracellular calcium concentration [Ca²⁺]ᵢ in cortical cells after stimulation with FeSO₄. A. The data show a representative fura-2 ratio (F340/F380) recorded from a cortical cell (circled) stimulated with FeSO₄ (40 μM; dotted curve), which does not occur with saline (solid curve). B. The change in fura-2 ratio [difference between basal (pre-stimulation) and maximal (stimulation)] stimulated by FeSO₄ was significantly greater than with saline (*p<0.05 by unpaired Student's t-test). The fura-2 ratio reflects the level of [Ca²⁺]ᵢ. Results are the means ±S.E.M of the number of the animals in parentheses. The arrow (A) indicates the time of FeSO₄ stimulation.

FIG. 12. A. As compared with the control eye receiving an implantation of an acrylate chip, the implanted iron particle caused a severe disorganization of the whole retina and an obvious loss of the photoreceptor outer segment (OS) 28 days after an iron implant. B. The antioxidant activity of the superoxide dismutase (SOD) was evaluated in the whole retinal extracts isolated from the control (acrylate chip) or the experimental (iron particle) eyes at 24 hours after implantation. The antibody against manganous superoxide dismutase (MnSOD) or β-actin respectively identified a 24 kDa or a 42 kDa band. The Western blot is a representative one from three independent experiments. C. Effect of the arylate (control) or the iron (experimental) on the immunoreactivity change of the superoxide dismutase (SOD) in the rat retina 24 hours after implantation. Respective phase contrast images are shown above. Consistent with the Western blot results, as compared to the control retina, there was an increase in SOD immunoreactivity. The increased immunoreactivity was localized in the outer retina, in particular the retinal pigment epithelial layer and the ONL. The abbreviations are: ONL, outer nuclear layer; OPL, outer plexiform layer; INL, inner nuclear layer; IPL, inner plexiform layer; GCL, ganglion cell layer. Scale bar=25 μm. D. Top curve. Electroretinogram (ERG) of a siderotic eye (FeSO₄ was injected five days earlier). Bottom curve. ERG of the normal fellow eye (untreated). In the siderotic eye both the a-wave and the b-wave amplitudes were dramatically reduced. In this particular example the b-wave ratio of the two eyes was only 30.70%. In addition, the a-wave ratio was 40.56%. E. Compared with the pre-siderotic a-wave amplitude ratio (0 day), the 25 nmole FeSO₄-induced reduction in the ratio was respectively nearly significant 3 days (p=0.0587) or significant 5 days after iron injection (*p<0.05; paired Student's t-test).

FIG. 13. The putative mechanism for iron-induced retinal toxicity.

FIG. 14. The effect of ferulic acid (FA) and tetramethylpyrazine (TMP) on rats given experimental siderosis. A. Compared with the pre-siderotic b-wave amplitude ratio (0 day), the 25 nmole FeSO₄-induced reduction in the ratio was significant 3/5 days after iron injection (*p<0.05; paired Student's t-test). In a dose dependent manner, TMP tended to attenuate, and FA significantly attenuated iron-induced reduction in b-wave amplitudes 3/5 days after siderosis when compared to saline-treated animals (†p<0.05; one-way ANOVA followed by Tukey multiple-comparison test). The results are the mean ±S.E.M. for the number of animals shown in the parenthesis. B (DAPI) and C (ChAT). The pictures reflected the effect of an intravitreous injected FA on the ChAT immunoreactivity affected by i.v.i. FeSO₄ (iron) after 5 days. The retinal cellular nuclei were counterstained with DAPI (blue colour; B1, B2, and B3). In the normal retina, ChAT immunoreactivity [red colour (conjugated with rhodamine)] is associated with star-shaped amacrine perikarya (short arrows) in the INL and GCL and their neuronal processes in the IPL, seen as two clearly defined strata (long arrows; C1). Iron caused an obvious obliteration of ChAT-labelled neuronal processes in the IPL (i.e. loss of the 2-band) and the number of ChAT immunoreactive amacrine cell perikarya and total DAPI-counterstained retinal nuclei was obviously reduced (picture not shown due to its similarity to the following picture C2). Presently, the siderotic insult was not affected by pre-siderotic administration of i.v.i. saline (5 μl; C2). In contrast, similar to the picture C1, the iron damage was nullified by i.v.i. 0.5 nmole FA given 60 minutes before the siderosis (C3). The corresponding DAPI-counterstained images of B1, B2, and B3 are respectively shown in picture C1, C2, or C3. The abbreviations are: ONL, outer nuclear layer; OPL, outer plexiform layer; INL, inner nuclear layer; IPL, inner plexiform layer; GCL, ganglion cell layer; ChAT, choline acetyltransferase; DAPI, 4,6-diamidine-2-phenylindole dihydrochloride. Scale bar=25 μm.

### DETAILED DESCRIPTION OF THE INVENTION

Acutely raising the intraocular pressure (IOP) of a rat eye, followed by reperfusion, is known to cause physiological dysfunction, the formation of oxygen free radicals and the death to RGCs and cholinergic amacrine cells as described. The present salicylate trapping assay has indicated that retinal ischemia induced by high IOP (HIOP) for 45, 60, and 75 minutes respectively resulted in mild, moderate and severe oxidative stress. The 60-minute HIOP-induced retinal ischemia was presently selected to evaluate the .OH scavenging activity of FA and TMP against moderate oxidative stress.

In the present invention, whether FA and TMP are able to protect against retinal ischaemia/experimental glaucoma induced by raising IOP for 60 minutes were investigated. Overall, this would provide evidence to support the view that therapies unrelated to lowering the IOP might be useful and help to improve physiological function, as well as reverse the pathological and molecular changes. The former is assessed in this invention by measuring the attenuation in the reduced ERG b-wave amplitude and indexation of the retinal ischaemia (Block, F., et al. Gen Pharmacol. 30(3):281-7, 1998). The latter is estimated by analyzing alleviation of inner retina damage to, for example, amacrine cells and RGCs (Osborne, N. N., et al. Neurochem Int. 29(3):263-70, 1996). Quantification of changes in the levels of ChAT/Thy-1 by immunoreactivity and in the mRNA levels of Thy-1 was also carried out. In this context, the aim of this invention was to confirm whether FA or TMP can protect retinal neurons against the ischaemic insult and whether the action is elicited via inhibition of .OH formation.

As mentioned, the present invention provides an approach for preventing against and managing with retinal ischemia or glaucoma comprising administering to a subject an effective amount of ferulic acid or its pharmaceutically acceptable salt, ester, solvate, hydrate, analogs, metabolite, enantiomer, isomer, tautomer, amide, derivative or prodrug. The ferulic acid or its prodrug reduces the b-wave of electroretinogram (ERG), inhibits the drastic reduction in the levels of Thy-1 mRNA and protein, and inhibits the formation of .OH.

Another oxidative stress experiments were carried out intraocularly in vivo. An iron particle (2 mm, 7.5 mg)/3 μl FeSO4 (24 mM, 8 mM, and 0.8 mM), or an acrylate chip (2 mm)/saline (3 μl) was intravitreously administered into one eye of a Wistar rat, the first two being the experimental treatments and the latter two being the respective controls. The final sham operation control was a needle inserted intra-jocularly and then removed.

The aim of the 2nd invention was to evaluate the oxidative stress effect of iron implant (chronic) or FeSO4 (acute) and the protective effect against iron-induced oxidative stress of putative neuroprotectants. The latter consisted of, as defined, two active compounds present in the Chinese herbal medicine Chuan Xiong, namely ferulic acid (FA) and tetramethylpyrazine (TMP).

The prevent invention provides a method for preventing or treating retinal or brain disease comprising administering an effective amount of ferulic acid, tetramethylpyrazine or their pharmaceutically acceptable salt, ester, solvate, hydrate, analogs, metabolite, enantiomer, isomer, tautomer, amide, derivative or prodrug to a subject. The said retinal disease comprises retinal ischemia, oxidative stress of the retina, glaucoma, age-related macular degeneration, or ocular hemorrhage, as well as brain ischaemia (i.e. stroke, infarction typed).

The effective amount of ferulic acid or tetramethylpyrazine is 0.1-25 nmole/kg. In a preferred embodiment of this invention, the effective amount is 0.5-5 nmole/kg. In a more preferred embodiment of this invention, the effective amount is 1-2 nmole/kg. The said subject is mammalian. In a preferred embodiment of this invention, the subject is human being. The effective amount of ferulic acid or tetramethylpyrazine can be in form of an eye drops, a solution, a syrup, a tonic, dry matter, powder, granules, or a tablet or capsule containing dry matter, powder, or granules. In a preferred embodiment of this invention, the effective amount of ferulic acid or tetramethylpyrazine is in form of an eye drops.

The ferulic acid, tetramethylpyrazine or their prodrug inhibits the formation of retinal/barin free radical. The said free radical is hydroxyl radical induced by raising intraocular pressure or cerebral ischaemia. The ferulic acid, tetramethylpyrazine or their prodrug not only inhibits the drastic reduction in the levels of Thy-1 mRNA and the Thy-1 immunoreactivity, indexing retinal ganglion cells but also inhibits the drastic reduction in the ChAT immunoreactivity, an index of amacrine cells. Also, it reduces the b-wave of electroretinogram (ERG).

The present invention further provides a method for preventing or treating iron-related disorder comprising administering an effective amount of ferulic acid, tetramethylpyrazine or its pharmaceutically acceptable salt, ester, solvate, hydrate, analogs, metabolite, enantiomer, isomer, tautomer, amide, derivative or prodrug to a subject. The said iron-related disorder is retained intraocular iron, brain hemorrhage (stroke, hemorrhagic type) or Alzheimer disease.

The effective amount of ferulic acid or tetramethylpyrazine is 0.1-25 nmole/kg. In a preferred embodiment of this invention, the effective amount is 0.5-5 nmole/kg. In a more preferred embodiment of this invention, the effective amount is 1-2 nmole/kg.

The said subject is mammalian. In a preferred embodiment of this invention, the subject is human being. The way for administering an effective amount of ferulic acid, tetramethylpyrazine or its prodrug comprises administering an eye drops, a solution, a syrup, a tonic, dry matter, powder, granules, or a tablet or capsule containing dry matter, powder, or granules. In a more embodiment of this invention, the way for administering to a subject is administering an eye drops.

The ferulic acid, tetramethylpyrazine or its prodrug inhibits the formation of retinal/barin free radical. The said free radical is hydroxyl radical induced by ferrous iron. Also, the ferulic acid or its prodrug inhibits the drastic reduction in the ChAT immunoreactivity, an index of amacrine cells and reduces the b/a-wave of electroretinogram (ERG).

### EXAMPLES

The methods were utilized to evaluate whether the defined agents could firstly ameliorate the detrimental physiological, biochemical, molecular biological and histopathological ischaemic/siderotic oxidative stress effects and, secondly, to identify the mechanism by which the damaged cells in the ischaemic/siderotic retina were protected.

### Example 1

### Induction of Retinal Ischaemia in a Rat

### 1. Animals

All investigations involving the use of animals conformed to the Association for Research in Vision and Opthalmology (ARVO) Statement for the Use of Animals in Opthalmology and Vision Research and were approved by Institutional Review Committee in Taipei Veterans General Hospital. The Wistar rats were bred at the animal house in Taipei Veterans General Hospital where the humidity was 40%-60% and the temperature 19° C.-23° C. They were kept on a 12 hour light/dark cycle with 12 to 15 air changes/hour. The animals were provided with food and water ad libitum.

### 2. Analgesia/Anaesthesia and Euthanasia of Animals

Wistar rats were anaesthetized with an intramuscular injection of ketamine (100 mg/kg) and xylazine (5 mg/kg). This combination provided sufficient analgesia/anesthesia to allow at least 60 minutes of surgery on the animals and then a rapid recovery. A half dose of both anesthetics was needed for the flash ERG recording in rats. The animals' perception of pain/discomfort and vital sign changes were closely monitored during the experimental procedure and recovery period with the body temperature being 36° C.-40° C., the heart rate being 250-450/minute and the respiration rate being 70-115/minute. A humane method listed in Schedule 1 of The Animals (Scientific Procedures Acts 1986) was used to kill the animals. Wistar rats were killed with an overdose (at least 140 mg/kg) of sodium pentobarbitone injected into the intraperitoneal cavity.

### 3. Induction of Retinal Ischaemia in Rats

Six-week old Wistar rats (body weight: 250˜500 g) were anaesthetized with an intramuscular injection of ketamine and xylazine as described above and placed in a stereotaxic frame. The anterior chamber of one eye, with the other serving as control, was cannulated with a 30-gauge needle connected to a 0.9% saline reservoir; this caused an increase in IOP to 120 mmHg. A whitening of the iris and the fundus of the retina confirmed induction of an ischaemic insult (Block, F., et al. Gen Pharmacol. 30(3):281-7, 1997). Animals were kept normothermic with a heated water jacket after the initial anaesthesia and were placed on a heating pad during recovery (both at 37° C.).

### Example 2

### Effect of 120-Minute Retinal Ischaemia on 2,3-dihydroxybenzoic Acid (DHBA) Formation: In Vivo Microdialysis

Rats were anesthetized and placed in a stereotaxic frame. This approach was adapted from Louzada-Junior et al with further modification. The microdilaysis tube was inserted into the vitreous cavity through the nonvascular pars plana of the sclera (0.5 mm from the corneal limbus) after it had been pre-punctured with a 25-gauge needle. This avoided the problem of bleeding into the vitreous cavity with consequent contamination of vitreous. With the microdialysis probe in position, the whole retina was subjected to HIOP-induced ischaemia for 120 minutes.

Trapping of .OH (Liu, D., et al. Free Radic. Biol. Med. 34:64-71, 2003; Chang, A. Y., et al. Exp. Neuro. 195:40-48, 2005) was accomplished by perfusing physiological saline containing 5 mM salicylic acid through a microdialysis probe (CMA/12, PC 14/01, CMA/Microdialysis, Stockholm, Sweden) inserted into the vitreous cavity of each rat's eye before and after the ischaemia. Salicylate was infused at the rate of 1.1 μl/min using a 1-ml Exmire microsyringe attached to a microdialysis pump (CMA-100, CMA/Microdialysis, Stockholm, Sweden). After an equilibration period of 45 min, a stable baseline was obtained. HIOP-induced retinal ischaemia was then performed. Samples were collected every 15 minutes starting 45 minutes before ischaemia and continuing for the subsequent 120-minute ischaemia. The microdialysates collected from the microdialysis probe were deposited into polyethylene vials (Microbiotech, Stockholm, Sweden) containing 15 μl of 0.1 M perchloric acid and were stored at −70° C. until analysis. The formation of .OH was quantified as the increase in 2,3-DHBA, which is the hydroxylative product of salicylic acid. Since 2,5-DHBA can also be produced via enzymatic pathways by virtue of P-450, 2,3-DHBA is considered to be a specific marker of .OH. Eluted microdialysates were directly injected into and 2,3-DHBA was analyzed by a high performance liquid chromatograph coupled with an electrochemical detector (HPLC-EC; Model LC-4C, Bioanalytical System Inc., West Lafayette, IN, USA). The detector output voltage (signal) was monitored using one data system (CSW 32, DataApex, CosmoBio, Czech). Samples were eluted at a flow rate of 0.8 ml/min. The HPLC-EC system was consisted of a CMA/200 refrigerated micro-sampler (20 μl loop), a solvent delivery system (BAS, PM-80) and an Alltima C18 column (5-μm particle size; 250 mm×4.6 mm i.d.; Alltech, Ill., USA). Isocratic separation was achieved at 4° C. A high-efficiency pulse damper was incorporated into the system to reduce background noise.

The DHBA compound was oxidized using a glassy carbon working electrode (oxidation potential: +750 mV; model MF 1000, Bioanalytical System Inc., West Lafayette, IN, USA) and an Ag/AgCl reference electrode (model MW 2021, Bioanalytical System Inc., West Lafayette, IN, USA) with a special thin-layer gasket for the microbore LC-EC. In order to minimize dead volume, the column was connected directly between the micro-sampler and the working electrode. The mobile phase included 1-heptane sulfonic acid sodium salt (MW: 202.25) 3.15 g, EDTA 0.15 g, phosphoric acid 4.5 ml, triethylamine 5.25 ml, acetonitrile 255 ml, distilled water (added to 1.5 liters), pH 2.7. Before use, the mobile phase was filtered through a 0.22 μm filter (Millipore, Billerica, Mass., USA) using vacuum assistance. The .OH were quantified using a standard curve, namely 12, 24, 36, 48 and 60 pmoles of 2,3-DHBA (which is photosensitive and can be dissolved in ethanol). This is the first invention utilizing the defined modified microdialysis system for the measurement of .OH in the rat eye in vivo.

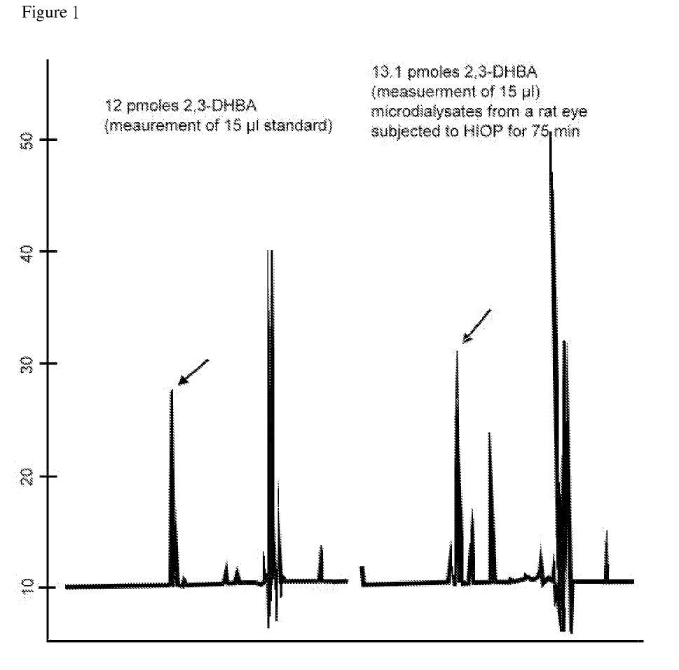

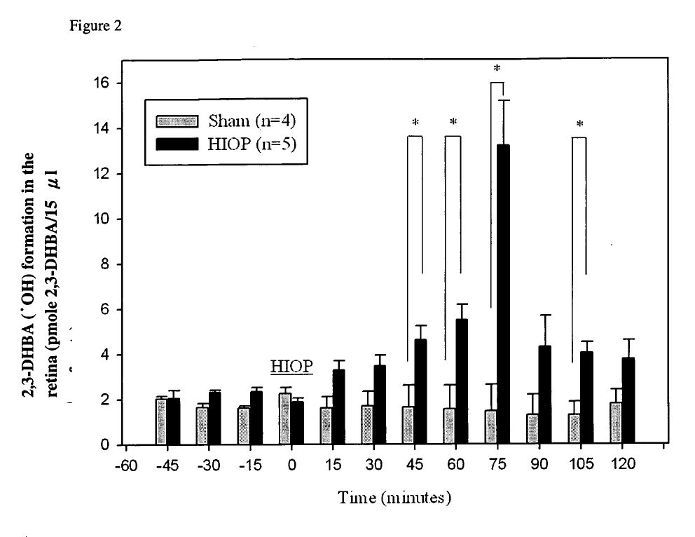

The production of .H was measured by establishing a standard curve utilizing different amounts of pure 2,3-DHBA (12-60 pmoles), for example 12 pmoles in FIG. 1A. Generally, the relevant peak representing 2,3-DHBA (hydroxylative products of salicylic acid) appeared at 18˜21 minutes in the HPLC chromatogram (arrows in FIG. 1 left and right). As shown in FIG. 2, the basal levels of 2,3-DHBA content in the vitreous dialysate samples following the intravitreous perfusion of salicylic acid in the sham operation rats were not different from those in ischaemic rats. As compared with the sham operation group, during the 120 minutes of retinal ischaemia, the levels of 2,3-DHBA and therefore .OH gradually increased, reaching a peak at 75 minutes (FIG. 2). As shown in FIG. 1 right, 13.1 pmoles of 2,3-DHBA was formed from 15 μl of a vitreous dialysate sample at 75 minutes of ischaemia. Furthermore, in comparison with the levels of 2,3-DHBA (pmole/15 μl perfusate) in the sham operation rats (n=4; 1.6296±0.9668, 1.5510±1.0459, 1.4728±1.1732, and 1.2845±0.5806 at 45, 60, 75, and 105 minutes of sham operation, respectively), the increases were statistically significant at defined 4 time-points of HIOP-induced retinal ischaemia (n=5; 4.6155±0.6169, 5.4964±0.6771, 13.1950±1.9699, and 4.0222±0.4819, respectively). However, in comparison with the levels of 2,3-DHBA (pmole/15 μl perfusate) in the sham operation rats (n=4; 1.2881±0.9183), an increase with no significance was found at 90 minutes of ischaemia (n=5; 4.2770±1.3837).

### Example 3

### Drug Administration

For electro-physiological, immuohistochemical, and molecular biological studies, drug administration was carried out with pre-administration (60 minutes before HIOP) of vehicle (saline for FA/TMP), FA (0.5 or 0.1 nmole), or TMP (0.5 nmole). The 60-minute HIOP procedure to induce ischaemic damage was carried out as defined. Intra-vitreous injection (i.v.i.) of 5 μl of each compound was given to the ‘ischaemic’ eye of each test rat using a 30-gauge needle attached to a 20 μl Hamilton syringe. As a control, the same volume of vehicle was intravitreously injected into the “ischaemic” eye of each control rat. In both cases, the fellow untreated eye acted as a normal control eye. In this case, FA, TMP, or vehicle was applied only to the eye (ipsilateral) which received ischaemia. Dose-response experiments for FA will also be performed.

### Example 4

### Recording of Flash ERG

The animals were first dark adapted for at least 8 hours, anaesthetized as already described and the pupils dilated with 1% tropicamide and 2.5% phenylephrine. The animal was then placed in a stereotaxic frame and the body temperature maintained at 37° C. with an electrical blanket. A platinum wire loop was placed on the corneal surface to act as the recording electrode. A ground electrode was connected to the scruff of the back together with a reference electrode to the tongue of the animal. A strobe was placed 5 cm in front of the animal to provide a stimulus of 0.5 Hz. Fifteen consecutive responses were recorded at two-second intervals and at 10 kHz; the responses were amplified and averaged using an amplifier P511/regulated power supply RPS107/stimulator PS22 (Grass-Telefactor, Astro-Med. Inc., R1, USA). The a-wave amplitude was measured from the baseline to the trough of the a-wave and the b-wave amplitude was measured from the trough of the a-wave to the peak of the b-wave.

However, variations were found to exist between the normal b-wave amplitudes in the various animals and therefore, for comparative purposes, the b-wave ratio [the b-wave amplitude of the ischaemic/sham operation eye compared with that of the contralateral normal (control) eye] was calculated and used in this invention.

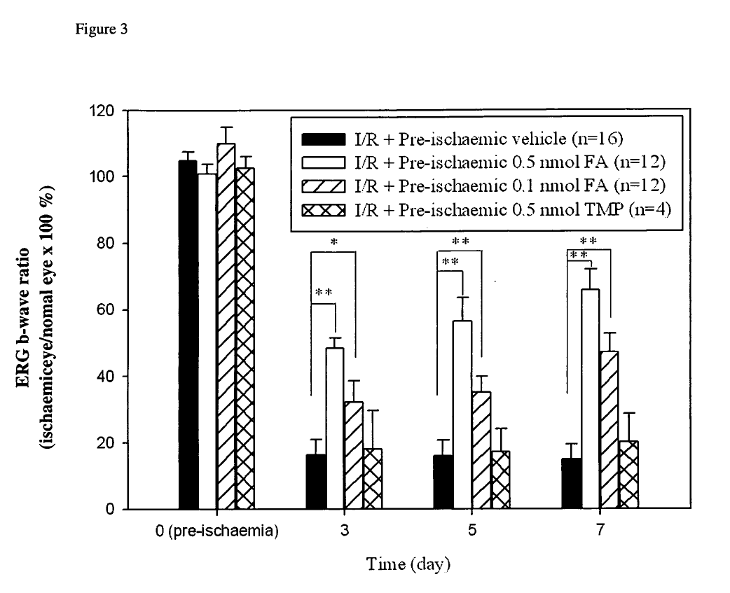

As shown in the FIG. 3, in comparison with the pre-ischaemia b-wave ratio baseline (0 day; n=16; 104.8553±2.6866), after I/R (induced by 60 minutes HIOP), there was a significant reduction in the ERG b-wave amplitude in the ischaemic retina pre-treated with vehicle. Moreover, the ischaemia-reduced b-wave amplitude was irreversible on 3^{rd} (16.4438±4.4678), 5^{th} (16.0258±4.7506), or 7^{th} day (14.8545±4.5586) after I/R. What is more, as compared with the vehicle-pre-treated rats, the pre-treatment of FA [0.5 nmoles (n=12); 0.1 nmoles (n=12)] significantly and dose-dependently attenuated the ischaemia-reduced b-wave amplitude (ratio) at 3 (48.3576±3.1633; 32.2402±6.3012), 5 (56.4287±6.9891; 35.0948±4.6412), or 7 days (65.8600±6.0998; 47.0775±5.5396) after ischaemia/reperfusion. In contrast, at defined time-points (17.9250±11.7135 at 3^{rd} day I/R; 17.1500±6.8996 at 5^{th} day I/R; 20.1750±8.5027 at 7^{th} day I/R), the pre-treatment of TMP (n=4) only showed a slight and insignificant attenuating effect on the retinal physiological dysfunction caused by retinal ischaemia.

### Example 5

### Immunofluorescence Analysis

### 1. Isolation and Cryosection of a Rat Retina

On day 7 after retinal ischaemia pre-treated with vehicle or tested compounds, the Wistar rats were killed by an overdose of sodium pentobarbitone injected into the peritoneal cavity as mentioned earlier. Rat retinas were carefully and rapidly dissected from the eyecups with the aim of completely detaching the sensory retina from the RPEs. They were incubated in Mg²⁺-free Locke's buffer containing: 154 mM NaCl, 5.6 mM KCl, 2.3 mM CaCl₂, 3.6 mM NaHCO₃, 5 mM N-[2-hydroxyethyl]piperazine-N′-[2-ethanesulfonic acid] (HEPES) and 5.6 mM D-glucose. The Locke's solution was briefly equilibrated before use with a gas mixture of 95% O2 and 5% CO₂ at 37° C., pH 7.4. Retinal pieces were fixed for a further 45 minutes in 4% (w/v) paraformaldehyde in sodium phosphate buffer (0.2 M NaH₂PO₄.2H₂O; 0.2M Na₂HPO₄; pH 7.4) and then transferred to 30% sucrose in sodium phosphate buffer for cryosectioning. Retinas were orientated and similarly mounted on a metal stand using O.C.T. compound (Merck Ltd) and then promptly frozen with dry ice. In this way, sections from similar areas were produced and thus the retinal sections (10 μm) had approximately the same eccentricity, which allowed direct comparison. The frozen retinal sections created on a microtome (Leica CM1900, Leica Instrument Ltd, USA) were placed on gelatin-coated slides and stored at −20° C. until use. Further retinal pieces were used for immunohistochemical study and the reverse transcriptase polymerase chain reaction (RT-PCR) assay.

### 2. ChAT Immunofluorescence Analysis

Rats were intracardially perfused first with 0.9% saline and then with 4% (w/v) paraformaldehyde. Seven days after sham operation alone or retinal ischaemia pre-treated with vehicle or defined agents, retinal pieces were processed as described. Retinal sections were incubated with primary antibody, rabbit anti-ChAT polyclonal:antibody (1:500; AB143; Chemicon, Temecula, Calif., USA). After washing with phosphate buffer saline (PBS), retinal sections were then incubated with secondary antibody, rhodamine-conjugated affinity purified antibody (1:50; Chemicon, Temecula, Calif., USA). The nuclei of the cells were labeled by 4,6-diamidine-2-phenylindole dihydrochloride (DAPI; 30 nM; Molecular Probes, Eugene, Oreg., USA).

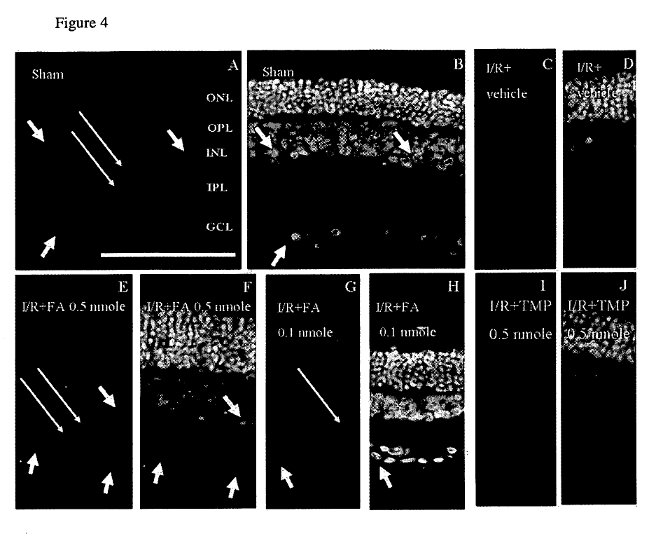

As revealed in FIG. 4, in the sham operation (normal) retina (A), ChAT immunoreactivity is associated with amacrine cell bodies (short arrows) found in the INL and RGC layer and with their processes in the IPL, which are seen as a two-band pattern (long arrows). Retina ischaemia plus reperfusion for 7 days caused an almost complete disappearance of ChAT immunoreactivity in the IPL and the number of ChAT immunopositive amacrine cell bodies was drastically decreased; the immunoreactive changes were not influenced by the pre-treatment of vehicle (C). The effect of ischaemia/reperfusion was obviously or partially counteracted by pre-administration of 0.5 nmole or 0.1 nmole FA, respectively (E: 2-band distribution of neuronal processes in the IPL and numerous ChAT-labeled amacrine cell bodies on either side; G: a single cell body in the RGC layer and strata pattern in the IPL); however, 0.5 nmole TMP had no effect (I: loss of neuronal process in the IPL and amacrine cell body on either side). The retinal cellular nuclei in different groups were counterstained with DAPI. The merged images for ChAT and DAPI labeling are shown in pictures B, D, F, H, and J. As indicated by the short arrows, DAPI-counterstained cellular nuclei (blue) in the normal (sham) or FA-treated ischaemic retina were confined within the range of ChAT-labeled amcrine cells (red; B, F and H). Moreover, as shown by the long arrow indicators, ChAT-labeled 2-band (red; B and F) or 1-band neuronal process distribution (H) was present in the IPL.

### 3. Thy-1 Immunofluorescence Analysis

This was repeated with another primary antibody, anti-Thy-1 monoclonal antibody (clone OX-7; Santa Cruz Biotechnology, Santa Cruz, Calif., USA; 1:100), a marker of RGCs, and in this case the secondary antibody was FITC-conjugated anti-mouse immunoglobulin (IgG) (Jackson ImmunoResearch, West Grove, Pa., USA; 1:100). After rinsing several times with PBS, the retinal sections were mounted using glycerol-PBS (1:1). Coverslips were placed on the slides, sealed and air dried. The retinal sections were examined using a confocal microscope. An independent scientist within the laboratory was asked to observe the change in the ischaemic retina pre-treated with vehicle or the test compounds in terms of immunolabeling compared to the sham operation (normal) retina.

As shown in the FIG. 6, Thy-1 is primarily associated with the RGC dendrites, bodies, and axons. This is illustrated in picture A where Thy-1 immunoreactivity is located over the IPL (dendrites) and RGC layer (axons/cell bodies). Thy-1 immunolabeling in the normal retinas subjected to sham operation is associated with a broad band in the ganglion cell layer (GCL) and IPL (A). A thinning of Thy-1 immunoreactivity can be observed 7 days after ischemia/reperfusion in the ischaemic retinas pre-administered with vehicle (B). In contrast, in the ischaemic retinas pre-treated with 0.5 nmole FA (C), the ischaemia-induced reduction in the thickness of Thy-1 immunoreactivity was less pronounced. As revealed in the bar chart (D), the vehicle-pre-treated ischaemic retinas (n=4; IPL+GCL thickness=34.3125±1.4156 μm) showed a significant reduction in the thickness of Thy-1 immunolabeling of the RGC layer plus the IPL by around 29%, in comparison to the control normal retinas (sham operation; n=4; IPL+GCL thickness=48.3750±4.1590 m). This ischaemia-induced reduction was significantly attenuated by around 15% with the treatment of 0.5 nmole FA (n=4; IPL+GCL thickness=41.62501.1250 μm).

### Example 6

### The Effect of Ferulic Acid on Retinal Ischaemia Revealed by RT-PCR

Levels of Thy-1 mRNAs present in the retinas were determined using a semi-quantitative RT-PCR technique. These were compared with the mRNA levels measured for the house-keeping gene β-actin as an internal standard (Nucci, C., et al. Invest Opthalmol V is Sci. 48(7):2997-3004, 2007). Seven days after sham operation alone or retinal ischaemia pre-treated with vehicle or defined chemicals, the rats were killed and the retinas removed and sonicated in TriReagent (as per manufacturer's instructions). Total retinal RNA was isolated and first strand cDNA synthesis was performed on 4 μg DNase-treated RNA (Nash, M. S., et al. Invest. Opthalmol. Vis. Sci. 40:1293-8, 1999). The PCR reaction was then initiated by an incubation at 94° C. for 10 min and this was followed by the required number of PCR cycles that ensured saturation had not been reached, but were suitable for comparison of cDNA levels in each sample (for β-actin: 24 cycles of 94° C., 40 s; 55° C., 40 s; 62° C., 60 s; for Thy-1: 34 cycles of 94° C., 40 s; 48° C., 40 s; 72° C., 40 s). Cycling was performed using a machine GeneAmp® PCR System 9700 (AB Applied Biosystems, CA, USA). After the final cycle was completed, there followed a final incubation of samples at 72° C. for 3 min.

The PCR oligonucleotide primers were obtained from Gibco Life Technologies (Paisley, Scotland, UK) and were listed at SEQ ID NOS: 1, 2, 3, and 4.

The PCR reaction products were separated on 1.5% agarose gels using ethidium bromide for visualization. The relative abundance of each PCR product was determined by analyzing photographs of the gels by SigmaScan (Jandel Scientific, Corte Madera, Calif.). One-way analysis of variance (ANOVA) was performed followed by the Tukey multiple-comparison test and a p value less than 0.05 was considered significant.

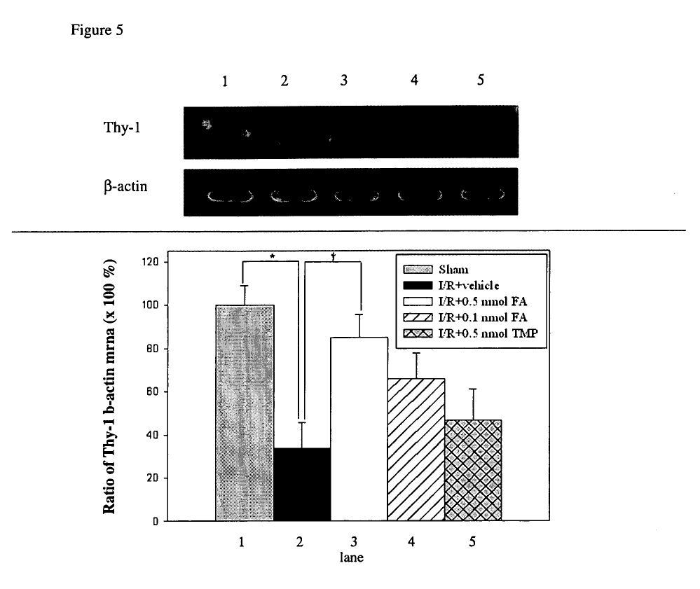

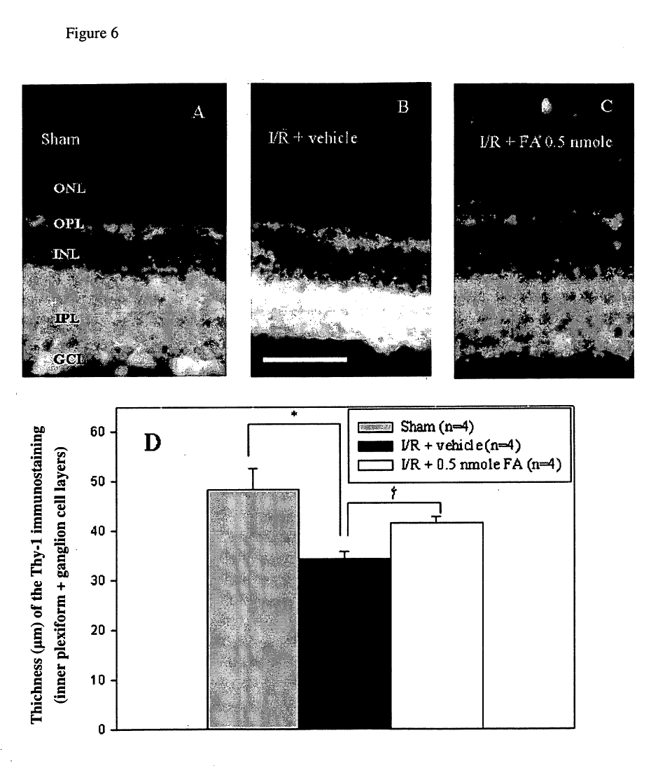

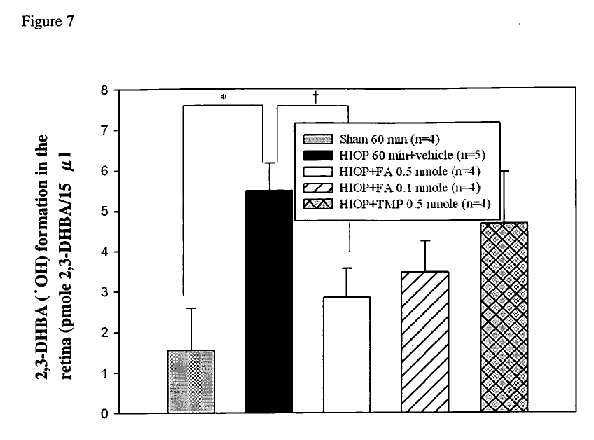

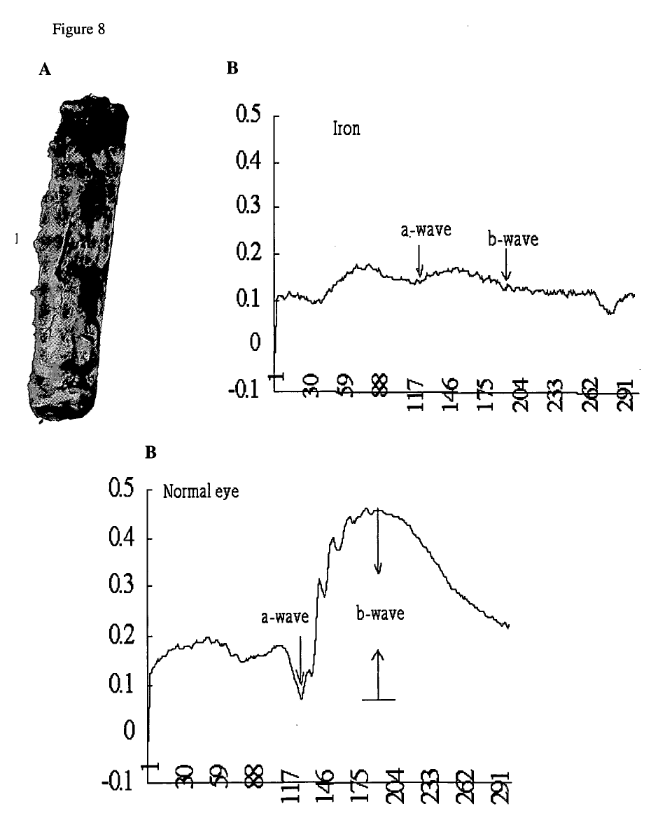

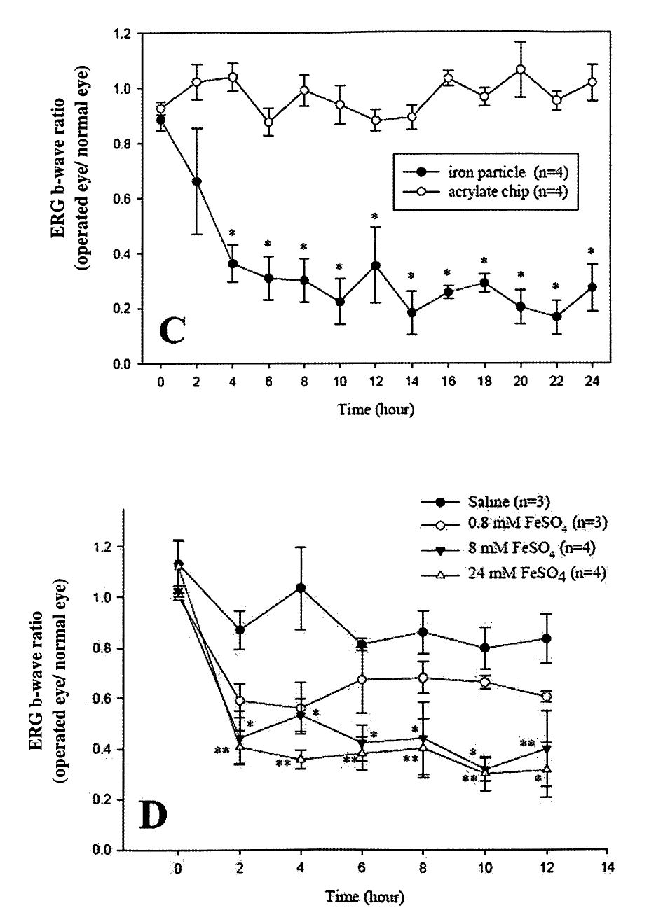

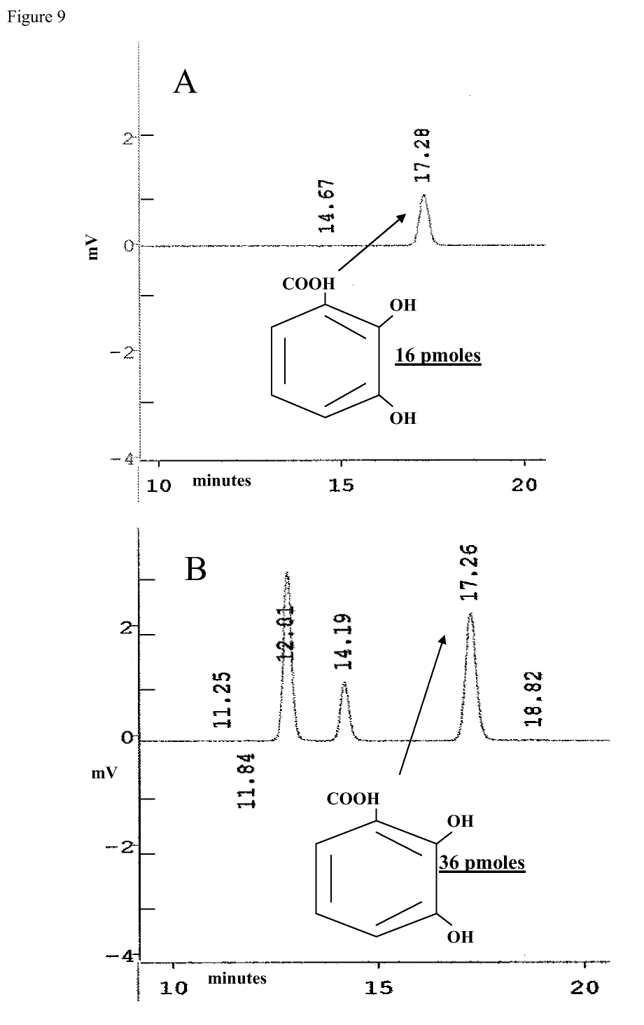

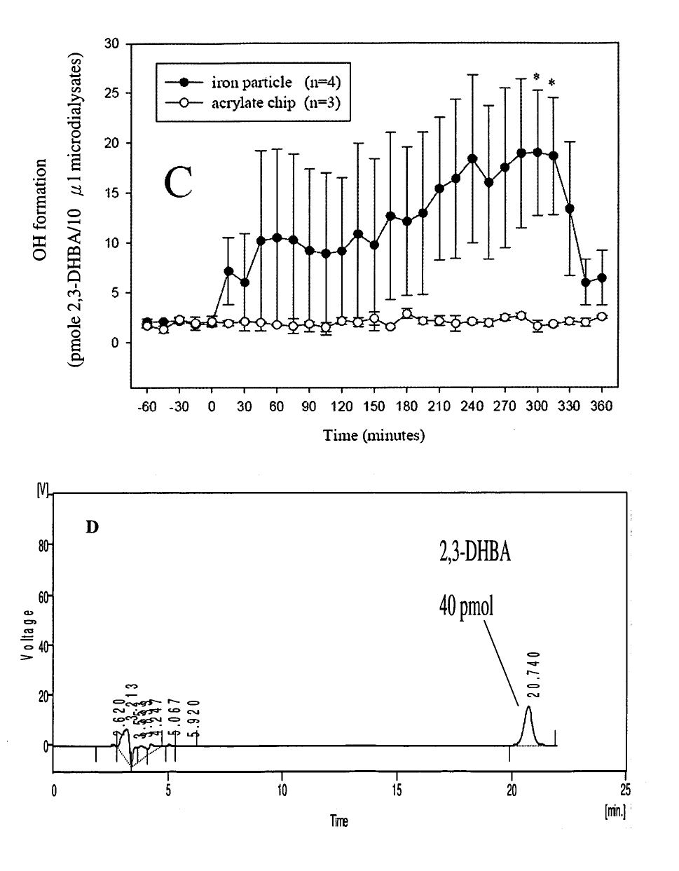

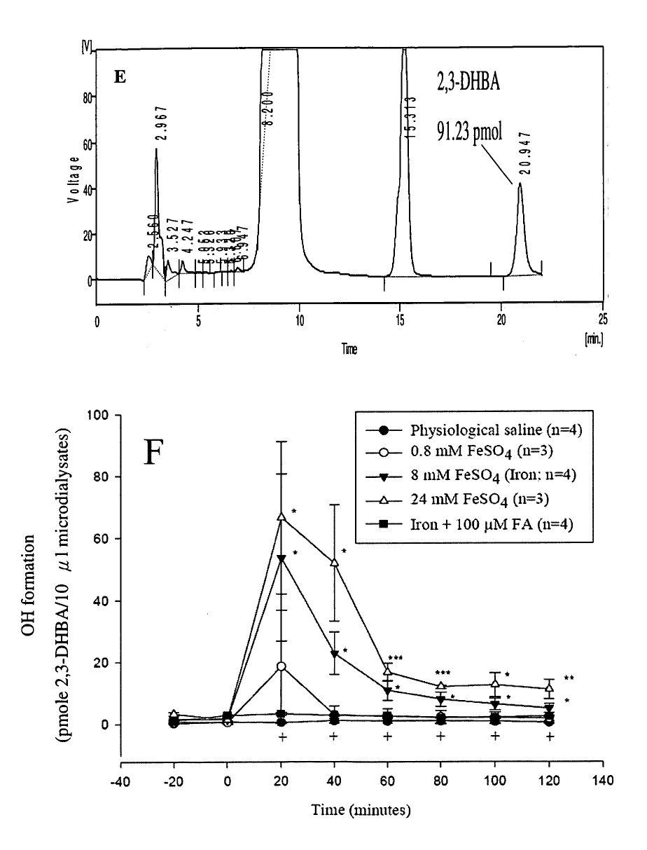

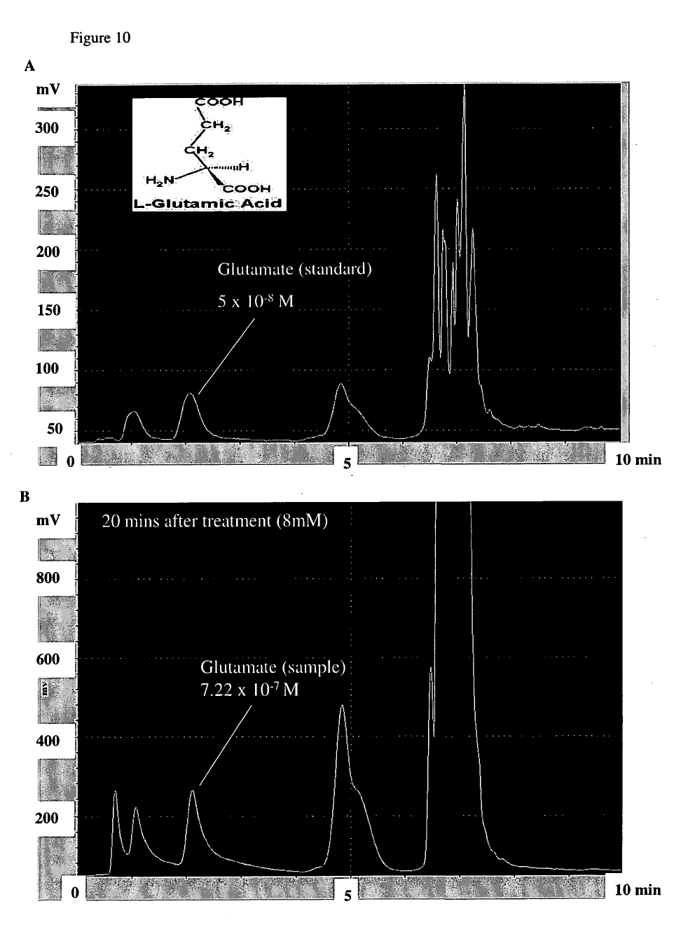

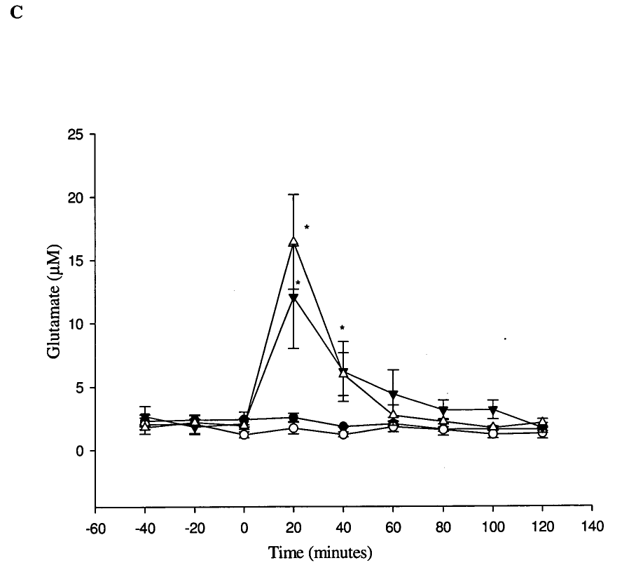

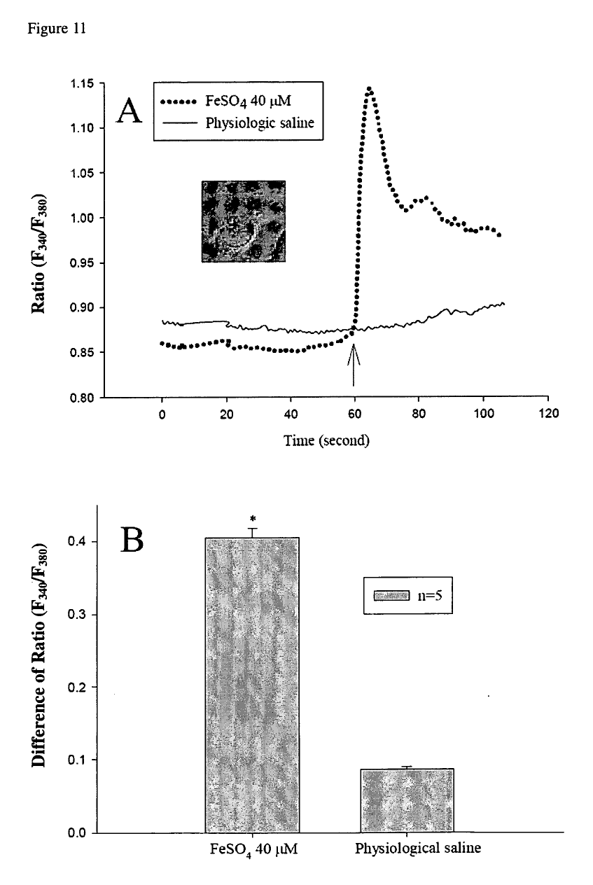

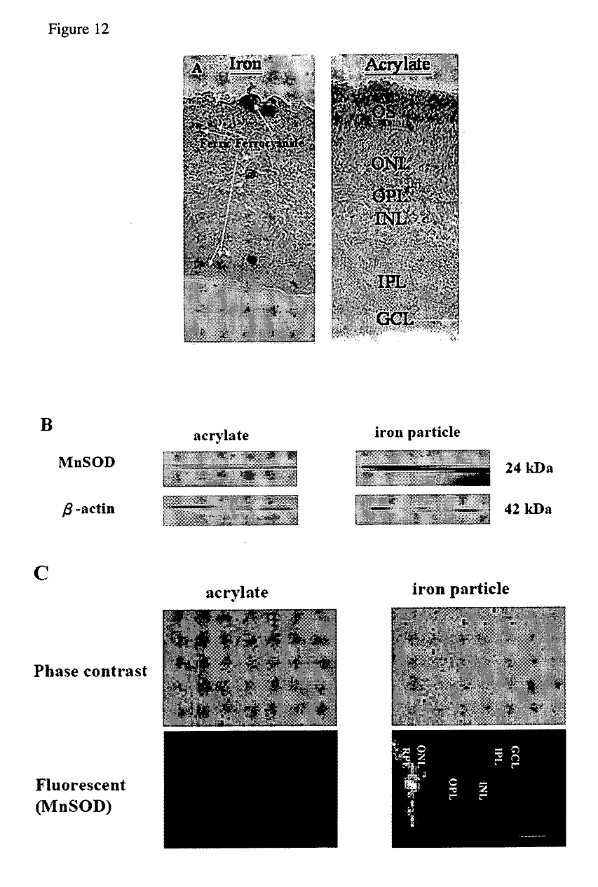

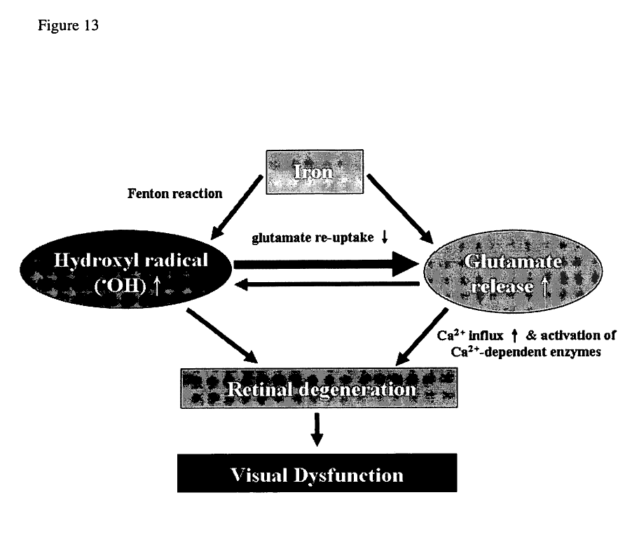

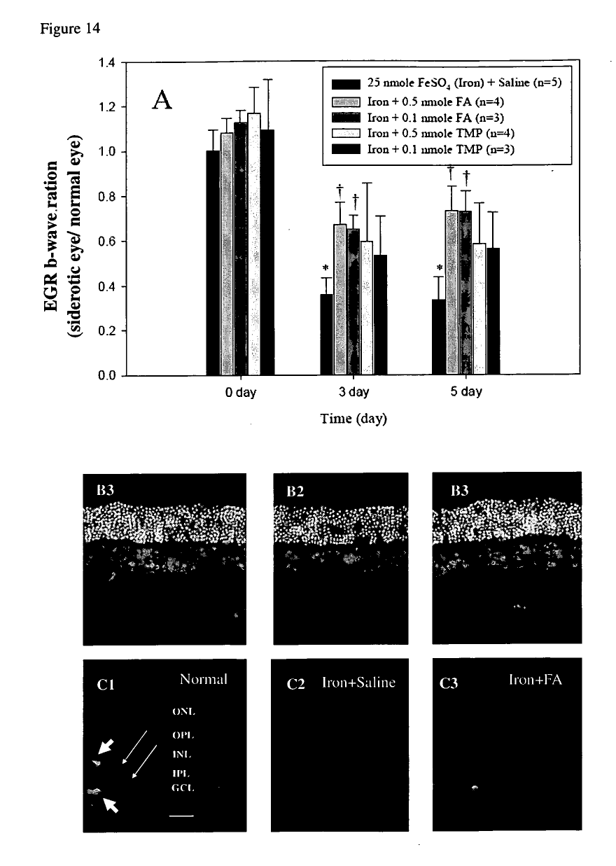

In FIG. 5, the minigel result (upper picture) suggests that expression of Thy-1 mRNA in the ischaemic retina pre-treated with vehicle (lane 2) was down-regulated as compared with that in the normal retina (sham operation; lane 1). This down-regulation was inhibited by pre-administration with 0.5 (lane3)/0.1 nmole ferulic acid (lane 4) and 0.5 nmole TMP (lane 5). Moreover, the inhibitory effect of 0.5 nmole FA was the greatest, followed by 0.1 nmole FA and then by 0.5 nmole TMP. Consistent with this, quantitative analysis (n=4; lower picture) showed that when the ratio of Thy-1 mRNA to β-actin mRNA was compared between the normal retina (sham operation; 100.0000±9.1287) and the vehicle-pre-treated ischaemic retina (33.7102±12.0285) there was a significant reduction by 66% (lower panel). Furthermore, the inhibitory effect of 0.5 nmole and 0.1 nmole FA as well as 0.5 nmole TMP on this ischaemia-induced reduction as measured by the Thy-1 mRNA/β-actin mRNA ratio was consistent with the minigel results and the order of effect by the different chemicals described above (85.1745±10.3177, 65.8968±11.9579, and 46.5876±14.4476, respectively). Furthermore, the inhibitory effect of 0.5 nmole FA was significant.

### Example 7

### The Effect of FA on the ROS In Vivo

As shown in the FIG. 7, at 60 minutes of sham operation or HIOP-induced retinal ischaemia, as compared with those from the sham group (n=4; 1.5510±1.0459), the measurements of the vitreous dialysates from the vehicle-pre-treated ischaemic eyes showed a significant (around 3.5 folds) increase in the levels of 2,3-DHBA (.OH; n=4; 5.4964±0.6771). Importantly, the pre-treatment of 0.5 nmole FA revealed a significant (around 2 folds) attenuation in the ischameia-evoked increased 2,3-DHBA levels (n=4; 2.8574±0.7186). In contrast, the pre-administration of 0.1 nmole FA (n=4; 3.4807+0.7707) or 0.5 nmole TMP (n=4; 4.6865±1.2661) only had a trend with no significance to attenuate ischameia-stimulated increase of .OH production.

### Example 8

### Induction of Iron-Induced Retinal Oxidative Stress in a Rat

### 1. Analgesia/Anaesthesia and Euthanasia of Animals

The steps were described in example 1.

### 2. Experimental Siderosis

The Wistar rats were anesthetized with an intramuscular injection of ketamine (100 mg/kg) and xylazine (5 mg/kg), and placed in a stereotaxic frame. The chronic model of experimental siderosis was established as follows. Through a port punctured by a 20-gauge intravenous catheter (0.5 mm from the corneal limbus), an autoclaved defined iron particle was slowly and carefully implanted into the vitreous cavity of one eye of each experimental rat through the catheter sheath by the catheter inner shaft (a needle). The punctured wound was then sutured with sterilized 10-0 nylon to keep a close system. Similarly (0.5 mm from the corneal limbus) and guided by the 20-gauge intravenous catheter, an autoclaved acrylate chip was implanted into the vitreous cavity of one eye of each control rat.

Similarly, the acute model of experimental siderosis was set up utilizing various concentrations of iron sulfate with the control group being injected with saline. The animals were kept normothermic with a heated water jacket after anesthesia and were placed on an electrical blanket during recovery (both set at 37° C.).

### Example 9

### Drug Administration

Iron toxicity and the formation of .OH were stimulated by an intravitreous application of FeSO₄ with or without the presence of the test compounds FA and/or TMP. For the dose-response iron toxicity experiments, 24 mM (72 nmoles), 8 mM (24 nmoles) and 0.8 mM (2.4 nmoles) of FeSO4 were utilized. To test drug biochemical effects on the oxidative stress caused by the experimental siderosis, an intravitreous injection of FA (100 mM) was carried out 60 minutes before siderosis. In further electro-physiological and immuohistochemical studies, with or without pre-administration of 0.5 or 0.1 nmoles of FA/TMP, a medium amount of FeSO₄ (25 nmole) was chosen to induce damage due to such as an oxidative stress so as not to reach the maximal pharmacological effect.

### Example 10

### Recording of Flash ERG

The steps of recording of flash ERG were described in example 4. The iron particle, after four weeks intraocular insertion into the rat's vitreous cavity, was obviously oxidized on retrieval (FIG. 8A). Notably, when the defined iron particle was retained intraocularly for 1 day, both a- and the b-wave amplitudes in the siderotic eye were drastically reduced compared to the normal untreated eye (FIG. 8B). Specifically, measuring every two hours (4˜24/2˜12 hours; FIGS. 8C and 8D) after intravitreous administration of an iron particle (n=5) or FeSO4 [8 mM (n=4); 24 mM (n=4)], respectively, the introduction was associated with a significant reduction in the ERG b-wave amplitudes compared to either the sham (n=3, data not shown) or control groups [acrylate (n=4), saline treatment (n=3)]. The reduction was greatest with 24 mM FeSO4, then with 8 mM, and least with 0.8 mM (FIG. 8D). Furthermore, when the iron and acrylate chips were compared in terms of b-wave ratio, the signal was reduced to about 65% at 2 hours (FIG. 8C). Similarly, with 8 mM FeSO4, the ration was, to a greater extent, reduced to about 45% at 2 hours (FIG. 8D).

Compared with the pre-siderotic a- or b-wave amplitude ratio (0 day), the 25 nmole FeSO4 significantly (p<0.05) induced a reduction in the a- (FIGS. 12D and 12E; n=5) or b-wave ratio (FIGS. 12D and 14A; n=5) at 3 and 5 days after iron injection. In a dose-responsive manner, TMP tended to alleviate, and FA significantly alleviated FeSO₄ induced reduction in the b-wave amplitudes at 3 and 5 days after siderosis compared to the saline-treated animals (FIG. 14A). FA was more effective that TMP.

### Example 11

### Reactive Oxygen Species (ROS) Assay In Vivo

Trapping of hydroxyl radicals was accomplished by perfusing physiological saline containing 5 mM salicylic acid through a microdialysis probe (CMA/12, PC 14/01, CMA/Microdialysis, Stockholm, Sweden) inserted into the vitreous cavity of a rat eye after intra-vitreous implantation of either an iron or acrylate chip for the defined chronic model and its control. A similar approach was used for the acute model and its control where, iron sulphate or distilled water was intra-vitreously injected with/without the drug to be tested. Salicylate was infused at the rate of 1.1 μl/min using a 1-ml Exmire microsyringe attached to a microdialysis pump (CMA-100, CMA/Microdialysis, Stockholm, Sweden). This was carried out every 15 or 20 minutes starting either 60 or 20 minutes before the implantation/injection and continued for 6 hours or 120 minutes following treatment, respectively. The formation of hydroxyl radicals was quantified as the increase in DHBA as the steps described in Example 2. The hydroxyl radicals were quantified by using 8, 16, 24, 32 and 40 pmoles of 2,3-DHBA (which is photosensitive and can be dissolved in ethanol) as an internal standard.

A standard curve was established for 2,3-DHBA (FIGS. 9A and 9D; for example 16 pmoles of 2,3-DHBA at FIG. 9A, or 40 pmoles at FIG. 9D). When the various intravitreous introductions were carried out, 36 pmoles and 91.23 pmoles of 2,3-DHBA (FIGS. 9B and 9E), were formed at 135 or 20 minutes after iron implant or injection (8 mM FeSO4), respectively. Generally, the peak appeared at 18˜21 minutes in the HPLC chromatogram (FIGS. 9A, 9B, 9D, and 9E) representing 2,3-DHBA, the hydroxylative products of salicylic acid as defined. Furthermore, as compared with the control groups, the intravitreous iron [particle (n=4); 8 mM FeSO4 (n=4); 24 mM FeSO4 (n=3)] all significantly enhanced the production of toxic .OH, at many time-points. The level of 2,3-DHBA stimulated by iron reached a peak around 300 minutes in the chronic model after a slow rise (FIG. 9C) but peaked at 20 minutes in the acute model after a rapid rise (FIG. 9F). Overall, compared to the saline treatment group, the iron-induced 2,3-DHBA levels rose dose-dependently and significantly in the acute model from 20 to 120 minutes after siderosis (FIG. 9F). As indicated by FIG. 9F, FA (100 and 20 mM; the latter not shown) dose-dependently and significantly decreased the formation of .OH compared with the controls (receiving 8 mM FeSO4 alone).

### Example 12

### Determination of the Levels of Glutamate

Using the same microdialysis system as the ROS assay in vivo, each microdialysate was assayed for the presence of derivatized glutamate product by HPLC with a fluorescence detector (FL; model CMA/280, CMA/Microdialysis, Stockholm, Sweden). The derivatization reaction was carried out at room temperature. O-phthalaldehyde (OPA; 27 mg; Fisher Scientific, Loughborough, UK) was dissolved in 1 ml methanol, 9 ml 0.4 M potassium tetraborate (MW: 303.53; borate buffer: 1.5 g borate plus pure water adjusted to pH 9.5 with 6 M NaOH and then added with water to 100 ml) and 5 μl β-mercaptoethanol (Fisher Scientific, Loughborough, UK). The OPA stock reagent was covered in foil and stored at −20° C. for 1 month. The OPA working solution was prepared each day by diluting 1 ml of the OPA stock reagent with 3 ml of potassium tetraborate (0.4 M). The derivatized agent (20 μl) was reacted with 20 μl of dialysate or glutamate standard for 2 min before injection onto the analytical column (Yang, C. H. et al., J Chromatogr B Biomed Sci Appl 734(1):1-6, 1999). For the HPLC separation, the detector output voltage (signal) was monitored using one data system (Scientific Information Service Corporation, Taiwan). Samples were injected onto the column and eluted at a flow rate of 0.8 ml/min. The HPLC-FL system consisted of a CMA/200 refrigerated micro-sampler (20 μl loop), a solvent delivery system (BAS, PM-80) and a Hypersil ODS column (5 μm particle size; 100×2.1 mm i.d., Thermo Fisher Scientific, Inc., Waltham, Mass., USA). Gradient separation was achieved at 4° C. A high-efficiency pulse damper was incorporated into the system to reduce background noise.

Before use, the mobile phase was filtered through a 0.22 μm filter (Millipore) using vacuum assistance. The linear gradient started with 100% mobile phase A [5% (v/v) acetonitrile, 0.55% acetic acid, 95% (v/v) sodium acetate buffer (11.5 ml glacial acetate added with 1800 ml deionised H₂O and titrated with 6 M NaOH to reach pH 6.0 and then mixed with deionised H₂O to 2 liter)] for 4 minutes, 100% mobile phase B [90% (v/v) acetonitrile, 0.058% acetic acid, 10% (v/v) sodium acetate buffer] was reached at time 5 minute and for 5 minute. At time 11 minute, mobile phase A was 100%. Using this protocol, glutamate was successfully separated. External standard solutions, containing 0, 5×10⁻⁸, 10⁻⁷, 10⁻⁶, 2×10⁻⁶, 10⁻⁵, 10⁻⁴ M standard glutamate (Sigma, St. Louis, Mo., USA), were run before and after each sample group. The glutamate stock solution (10⁻² M) was prepared in double deionised water and kept at 4° C. The detection sensitivity was 10 M. All standard samples and test samples were analyzed in duplicate. This is also the first invention utilizing this method for the rat eye in vivo.

A standard curve for glutamate was created (FIGS. 10A and 10B). Compared to the saline control group (n=4), in the retina, the presence of FeSO₄ resulted in a dose-dependent release of glutamate, which was significant at peaks occurring 20 minutes and 40 minutes after siderosis with 24 mM (n=6) and/or 8 mM FeSO₄ (n=4; FIG. 10C).

### Example 13

### Cytosolic Calcium Measurement

The cortical cells to be tested were placed on a cover slip and loaded with calcium ion sensitive dye by incubation with a final concentration of 5 μM of the acetoxymethyl ester form of Fura-2 for 30 min at 37° C. in the 1 ml loading buffer. The cover slip was washed three times with buffer containing 150 mM NaCl, 5 mM KCl, 2.2 mM CaCl₂, 1 mM MgCl₂, 5 mM glucose, 10 mM HEPES, pH 7.4. A micropipette was pulled from a glass capillary by a micropipette puller (P-97, Sutter Instrument Co.) and the tip was polished in a microforge (o.d. 2 μm, MF-83, Narishige). The micropipette was backfilled with the test compound (40 μM FeSO₄ or saline) using a fine tip syringe. The cover slip was mounted on an inverted microscope to measure single-cell fluorescence. The ejection mircropipette was positioned at a distance of 10 μm from a cell. A short pulse (0.5s) of the test compound was then applied to a single cell through the micropipette by a microinjection system (Picospritzer II, General Valve) with a pressure of 15 psi. The intracellular calcium concentration, [Ca²⁺]i, was calculated using the ratio between the fluorescence at 340 nm and at 380 nm (F340/F380). Changes in the fluorescence ratio represent the changes in the [Ca²⁺]i (Yang, D. M., et al. J Microsc 209: 223-227, 2003).

A digital calcium imaging assay using the Fura-2 fluorescence system was used to compare the saline treatment group (n=5) with the 40 μM FeSO₄ (n=5) group and there was a significant increase in the [Ca²⁺]i in the cortical cells (FIG. 11B). FIG. 11A shows the fura-2 fluorescence ratios (F340/F380) for various time points after treatment; a representative cortical cell was shown after stimulation with FeSO₄: saline was found to have no affect on the cell's [Ca²⁺]i.

### Example 14

### Immunofluorescence Analysis

### 1. Isolation and Cryosection of a Rat Retina

The steps of isolation and cryosection of rat retina were described in example 5. The time course of the experiments involved enucleation of the rat eyes and retrieval of the retinal pieces on day 1 (Western blot), day 5 (immunohistochemistry) and day 28 (histopathology) after experimental siderosis with/without each test compound.

### 2. Western Blot Analysis

Equivalent amounts of protein were separated on a 12% sodium dodecyl sulfate-polyacrylamide gel electrophoresis after denaturation and then transferred to nitrocellulose membrane (NC; Amersham Pharmacia Biotech., England). The membranes were blocked for 2 h at room temperature with blocking buffer (150 mM NaCl, 25 mM Tris-HCl, pH 7.6 and 0.005% Tween) containing 5% skimmed milk. The blots were then incubated overnight, at 4° C., with mouse monoclonal anti-beta-actin antibody, (1:4000, Zymed Laboratories, Inc., San Francisco, Calif., USA), rabbit anti-catalase antibody (1:8000, Calbiochem, Darmstadt, Germany) or rabbit polyclonal anti-magnanese superoxide dismutase (SOD) antibody (1:100; Upstate, Lake Placid, N.Y., USA). The primary antibodies were diluted in blocking buffer containing 1% skimmed milk. After three washes, the membranes were incubated for 2 h at room temperature with horseradish peroxidase conjugated goat anti-mouse IgG or anti-rabbit IgG (Amersham; 1:25000 dilution in blocking buffer containing 1% skimmed milk) and then developed using the enhanced chemiluminescent (ECL) analysis system (SuperSignal West Pico Chemiluminescent Substrate, Pirece, Rockford, Ill.). An x-ray film was exposed to the ECL treated membrane for 30-60 seconds and then developed.

### 3. Immunofluorescence Analysis

For immunohistochemical analysis, rats were intracardially perfused first with 0.9% saline and then with 4% (w/v) paraformaldehyde. A defined period after the experimental siderosis, retinal pieces were processed as described previously (Chao, H. M., et al., Brain Res 904(1):126-136, 2001; Wood, J. P. M., et al., Exp Eye Res 72:79-86, 2001). In order to observe the expression of ‘ROS-related enzymes’, retinal sections were incubated with primary antibody: rabbit polyclonal anti-rat manganese SOD (1:100; Upstate, NY) or rabbit anti-catalase (1:1000; Calbiochem, CA). After washing with PBS, retinal sections were then incubated with secondary antibody, Cy™3-conjugated donkey anti-rabbit IgG (1:100; Jackson ImmunoResearch Laboratories, INC, PA, USA). This was repeated with another primary antibody, rabbit polyclonal anti-rat ChAT (1:500; AB143; Chemicon, Temecula, Calif., USA) and in this case the secondary antibody was rhodamine-conjugated affinity purified secondary antibody (1:50; Chemicon, Temecula, Calif., USA). The nuclei of the whole number of cells were labeled by DAPI (30 nM). After rinsing several times with PBS, retinal sections were mounted using glycerol-PBS (1:1). Coverslips were put on the slides, sealed and air dried. The retinal sections were examined using a confocal microscope.

An independent scientist within the laboratory was asked to observe the change in the siderotic retina with/without the test compound in terms of labeling compared to the normal retina.

Based on the Western blot results and the immunohistochemical results, it is clear that the intravitreous implanted iron particle activated SOD (FIGS. 12B and 12C) but not catalase (data not shown) as part of the retinal cell's self-defense anti-oxidative enzyme system. This occurred at 24 hours after siderosis. Interestingly, the enhanced SOD immunoreactivity was mainly confined to the outer retina, particularly the retinal pigment epithelial (RPE) and the outer nuclear layers (ONLs).

On the other aspect, the retinal cellular nuclei were co-stained with DAPI for the analysis (FIG. 14 B1, B2 and B3). In the normal retina (FIG. 14 C1), ChAT immunoreactivity is specific to cholinergic amacrine neurones in the inner nuclear layer (INL) and ganglion cell layer (GCL; as indicated by short arrows) and to their neuronal processes in the inner plexiform layer (IPL); these appear as two well-defined bands (as indicated by long arrows).

Iron (25 nmole FeSO₄ for 5 days) was able to cause loss of 2-band ChAT immunoreactivity in the IPL. This was accompanied by a drastic reduction in the number of total DAPI-stained retinal nuclei and, in particular, ChAT positive amacrine neurons in the INL and GCL. These results were similar to the immunopathological findings for retinas subjected to FeSO₄ with treatment of saline (FIG. 14 C2). Furthermore, similar to the normal retina (FIG. 14 C1), these iron-induced changes were counteracted by 0.5 nmole FA given 60 minutes before the siderosis (FIG. 14 C3).

### Example 15

### Iron Histochemistry

Retinal pieces were prepared as described previously (Chao, H. M., et al., Brain Res 904(1):126-136, 2001; Wood, J. P. M., et al., Exp Eye Res 72:79-86, 2001). Each retinal section was incubated with 200 μl of mixed solution (7.4% HCl:2% potassium ferrocyanide=1:1) for 20 minutes and washed with PBS. It was then incubated with 200 μl of nuclear fast red for 20 minutes and washed with PBS. After rinsing with PBS, the slides of the retinal sections were prepared as described (Chao, H. M., et al., Brain Res 904(1):126-136, 2001; Wood, J. P. M., et al., Exp Eye Res 72:79-86, 2001) and examined under a light microscope. Again, an independent scientist was assigned to assess the alterations in the siderotic retina compared to the normal retina.

Analysis of the histopathological results shows that 28 days after chronic siderosis by an intravitreous iron particle, obvious retinal disorganization with massive loss of the photoreceptor outer segment was visible (OS; FIG. 12A); this did not occur with the acrylate chip control. These changes would seem to be associated with a wide-spread distribution of ferric irons throughout all retinal layers (FIG. 12A).

The unpaired Student's t-test was used when comparing two independent groups and paired Student's t-test when comparing paired groups. One-way analysis of variance (ANOVA) was performed to compare three or more independent groups. Following the one-way ANOVA, the Tukey multiple-comparison test was carried out in order to compare the control column (e.g. vehicle-treated ischaemic/siderotic rats) with all other columns (e.g. FA/TMP-treated ischaemic/siderotic rats).

1. A method for preventing or treating retinal or brain disease comprising administering an effective amount of ferulic acid, tetramethylpyrazine or their pharmaceutically acceptable salt, ester, solvate, hydrate, analogs, metabolite, enantiomer, isomer, tautomer, amide, derivative or prodrug to a subject,
wherein the retinal disease comprising retinal ischemia, oxidative stress of the retina, glaucoma, age-related macular degeneration, or ocular hemorrhage as well as brain ischaemia (i.e. stroke, infarction typed).

2. The method of claim 1, wherein the effective amount of ferulic acid or tetramethylpyrazine is 1-2 nmole/kg.

3. The method of claim 1, wherein the subject is human being.

4. The method of claim 1, wherein administering an effective amount comprises administering an eye drops, a solution, a syrup, a tonic, dry matter, powder, granules, or a tablet or capsule containing dry matter, powder, or granules.

5. The method of claim 1, wherein the ferulic acid, tetramethylpyrazine or their prodrug inhibits the formation of retinal/barin free radical.

6. The method of claim 5, wherein the free radical is hydroxyl radical.

7. The method of claim 6, wherein the hydroxyl radicals is induced by raising intraocular pressure or cerebral ischaemia.

8. The method of claim 1, wherein the ferulic acid, tetramethylpyrazine or their prodrug inhibits the drastic reduction in the levels of Thy-1 mRNA and the Thy-1 immunoreactivity, indexing retinal ganglion cells.

9. The method of claim 1, wherein the ferulic acid, tetramethylpyrazine or their prodrug inhibits the drastic reduction in the ChAT immunoreactivity, an index of amacrine cells.

10. The method of claim 1, wherein the ferulic acid, tetramethylpyrazine or their prodrug reduces the b-wave of electroretinogram (ERG).

11. A method for preventing or treating iron-related disorder comprising administering an effective amount of ferulic acid or its pharmaceutically acceptable salt, ester, solvate, hydrate, analogs, metabolite, enantiomer, isomer, tautomer, amide, derivative or prodrug to a subject,
wherein the iron-related disorder is retained intraocular iron, brain hemorrhage (stroke, hemorrhagic type) or Alzheimer disease.

12. The method of claim 11, wherein the effective amount of ferulic acid, tetramethylpyrazine is 1-2 nmole/kg.

13. The method of claim 11, wherein the subject is human being.

14. The method of claim 11, wherein administering an effective amount comprises administering an eye drops, a solution, a syrup, a tonic, dry matter, powder, granules, or a tablet or capsule containing dry matter, powder, or granules.

15. The method of claim 1, wherein the ferulic acid, tetramethylpyrazine or its prodrug inhibits the formation of retinal/brain free radical.

16. The method of claim 15, wherein the free radical is hydroxyl radical.

17. The method of claim 16, wherein the hydroxyl radicals is induced by ferrous iron.

18. The method of claim 11, wherein the ferulic acid, tetramethylpyrazine or its prodrug inhibits the drastic reduction in the ChAT immunoreactivity, an index of amacrine cells.

19. The method of claim 11, wherein the ferulic acid, tetramethylpyrazine or its prodrug reduces the b/a-wave of electroretinogram (ERG).


SEQUENCE LISTING (4 sequences)

SEQ ID NO: 1 - DNA, 22 bp, Artificial
  forward primer
gaaccgctcattgccgatagtg

SEQ ID NO: 2 - DNA, 23 bp, Artificial
  reverse primer
ttgtccctgtatgcctctggtcg

SEQ ID NO: 3 - DNA, 22 bp, Artificial
  forward primer
cgctttatcaaggtccttactc

SEQ ID NO: 4 - DNA, 22 bp, Artificial
  reverse primer
gcgttttgagatatttgaaggt